SEQUENCE LISTING (37 sequences)

SEQ ID NO: 1 - RNA, 23 nt, Mus musculus
uuuggcacuagcacauuuuugcu

SEQ ID NO: 2 - RNA, 106 nt, Mus musculus
ccaguaccaucugcuuggccgauuuuggcacuagcacauuuuugcuugugucucuccgcugugagcaaucauguguagugccaauaugggaaaagcgggcugcugc

SEQ ID NO: 3 - RNA, 25 nt, Mus musculus
uuuggcaaugguagaacucacaccg

SEQ ID NO: 4 - RNA, 75 nt, Mus musculus
accauuuuuggcaaugguagaacucacaccgguaagguaaugggacccggugguucuagacuugccaacuauggu

SEQ ID NO: 5 - RNA, 22 nt, Mus musculus
uauggcacugguagaauucacu

SEQ ID NO: 6 - RNA, 70 nt, Mus musculus
cuguguauggcacugguagaauucacugugaacagucucagucagugaauuaccgaagggccauaaacag

SEQ ID NO: 7 - RNA, 23 nt, Mus musculus
ucuuugguuaucuagcuguauga

SEQ ID NO: 8 - RNA, 89 nt, Mus musculus
cgggguugguuguuaucuuugguuaucuagcuguaugagugguguggagucuucauaaagcuagauaaccgaaaguaaaaauaacccca

SEQ ID NO: 9 - DNA, 1949 bp, Artificial Sequence
  183F-Atoh1-HA
gtaatctagtggttgtaggacctccaggagaaggcagctgacccctctgcagggtctgcaggctggagagtgtgactcctgtcctgtgtatggcactggtagaattcactgtgaacagtctcagtcagtgaattaccgaagggccataaacagagcagagacagatccgcgagcaccttggagctcctcacccctttctgcctagacctctgtttccaggggtgccagggtacaaagacctcctctgctccttccccagagggcctgttccagtaccatctgcttggccgattttggcactagcacatttttgcttgtgtctctccgctgtgagcaatcatgtgtagtgccaatatgggaaaagcgggctgctgcggccacgttcacctcccccggcatcccagggtctgtgtgtctcactggctcccaggcccatctggcttactgctgggtgaggagggtacagccctaccctggtgaacagccaggatccggaccatagtctggaccttgtgttaactgtgggaagagcgccctcctaaaaccaccctaactgcttcttcttcagcataggcttactggtctggctgctggaggcctcccaccatttttggcaatggtagaactcacaccggtaaggtaatgggacccggtggttctagacttgccaactatggtgtaagtgctgagctgctgaaggtctgcaccgtgccggaacctgccgatcaccaggaaggagaggggactcctgtctccagaccaccaggcagtggcagagggtgggcgctcgagtactaactggtacctcttcttttttttttgatatcctgcagaagcttgatatcgaattcgccaccatgtcccgcctgctgcatgcagaagagtgggctgaggtaaaagagttgggggaccaccatcgccatccccagccgcaccacgtcccgccgctgacgccacagccacctgctaccctgcaggcgagagaccttcccgtctacccggcagaactgtccctcctggatagcaccgacccacgcgcctggctgactcccactttgcagggcctctgcacggcacgcgccgcccagtatctgctgcattctcccgagctgggtgcctccgaggccgcggcgccccgggacgaggctgacagccagggtgagctggtaaggagaagcggctgtggcggcctcagcaagagccccgggcccgtcaaagtacgggaacagctgtgcaagctgaagggtggggttgtagtggacgagcttggctgcagccgccagcgagccccttccagcaaacaggtgaatggggtacagaagcaaaggaggctggcagcaaacgcaagggaacggcgcaggatgcacgggctgaaccacgccttcgaccagctgcgcaacgttatcccgtccttcaacaacgacaagaagctgtccaaatatgagaccctacagatggcccagatctacatcaacgctctgtcggagttgctgcagactcccaatgtcggagagcaaccgccgccgcccacagcttcctgcaaaaatgaccaccatcaccttcgcaccgcctcctcctatgaaggaggtgcgggcgcctctgcggtagctggggctcagccagccccgggagggggcccgagacctaccccgcccgggccttgccggactcgcttctcaggcccagcttcctctgggggttactcggtgcagctggacgctttgcacttcccagccttcgaggacagggccctaacagcgatgatggcacagaaggacctgtcgccttcgctgcccgggggcatcctgcagcctgtacaggaggacaacagcaaaacatctcccagatcccacagaagtgacggagagttttccccccactctcattacagtgactctgatgaggccagttacccatacgatgttccagattacgcttaggcggccgcc

SEQ ID NO: 10 - DNA, 5553 bp, Artificial Sequence
  pAAV2.1-CMV-eGFP3-sd-96-sa-WPRE
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcatcaacaagtttgtacaaaaaagcaggcttgtaaaacgacggccagtgagcgcgcgtaatacgactcactatagggcgaattgggtaccgggccccccctcgacgtaatctagaggatccctcgaggacctcctctgctccttccccagagggcctgttccagtaccatctgcttggccgattttggcactagcacatttttgcttgtgtctctccgctgtgagcaatcatgtgtagtgccaatatgggaaaagcgggctgctgcggccacgttcacctcccccggcatcccagggtctgtgtgtctcactggctccctggcccatctggcttactgctgggtgaggagggtacagccctaccctggtgaacagccaggatccctcgagtactaactggtacctcttcttttttttttgatatcctgcagaagcttgatatcgaattcctgcagcccgggggatccactagttctagagcggccgccaccgcggtggagctccagcttttgttccctttagtgagggttaattgcgcgcttggcgtaatcatggtcatagcacccagctttcttgtacaaagtggttgatagcttggatcgatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 11 - DNA, 2813 bp, Artificial Sequence
  pME-MCS-sda
ctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacacattgatgagcaatgcttttttataatgccaactttgtacaaaaaagcaggcttgtaaaacgacggccagtgagcgcgcgtaatacgactcactatagggcgaattgggtaccgggccccccctcgacgtaatctagaggatccctcgagtactaactggtacctcttcttttttttttgatatcctgcagaagcttgatatcgaattcctgcagcccgggggatccactagttctagagcggccgccaccgcggtggagctccagcttttgttccctttagtgagggttaattgcgcgcttggcgtaatcatggtcatagcacccagctttcttgtacaaagttggcattataagaaagcattgcttatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaataatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcaagctcatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 12 - protein, 9 aa, Artificial Sequence
  Viral hemagglutinin tag
YPYDVPDYA

SEQ ID NO: 13 - DNA, 50 bp, ARTIFICIAL SEQUENCE
  Primer
gcggtcgacgtaatctagaggatccctcgagtactaactggtacctcttc

SEQ ID NO: 14 - DNA, 52 bp, ARTIFICIAL SEQUENCE
  Primer
gcaagcttctgcaggatatcaaaaaaaaaagaagaggtaccagttagtactc

SEQ ID NO: 15 - DNA, 27 bp, ARTIFICIAL SEQUENCE
  Primer
gcactagtggttgtaggacctccagga

SEQ ID NO: 16 - DNA, 45 bp, ARTIFICIAL SEQUENCE
  Primer
tccagactatggtccggatcctggctgttcaccagggtagggctg

SEQ ID NO: 17 - DNA, 45 bp, ARTIFICIAL SEQUENCE
  Primer
cctggtgaacagccaggatccggaccatagtctggaccttgtgtt

SEQ ID NO: 18 - DNA, 27 bp, ARTIFICIAL SEQUENCE
  Primer
gcctcgagcgcccaccctctgccactg

SEQ ID NO: 19 - DNA, 38 bp, ARTIFICIAL SEQUENCE
  Primer
cgaattcgccaccatgtcccgcctgctgcatgcagaag

SEQ ID NO: 20 - DNA, 68 bp, ARTIFICIAL SEQUENCE
  Primer
cgcgcggccgcctaagcgtaatctggaacatcgtatgggtaactggcctcatcagagtcactgtaatg

SEQ ID NO: 21 - DNA, 46 bp, ARTIFICIAL SEQUENCE
  Primer
gggctcgagagcaaaaatgtgctagtgccaaacccgggaattcgtt

SEQ ID NO: 22 - DNA, 48 bp, ARTIFICIAL SEQUENCE
  Primer
ggggcggccgctttggcactagcacatttttgcttctaggtttaaacg

SEQ ID NO: 23 - DNA, 48 bp, ARTIFICIAL SEQUENCE
  Primer
gggctcgagcggtctgagttctaccattgccaaacccgggaattcgtt

SEQ ID NO: 24 - DNA, 50 bp, ARTIFICIAL SEQUENCE
  Primer
ggggcggccgctttggcaatggtagaactcacaccgtctaggtttaaacg

SEQ ID NO: 25 - DNA, 45 bp, ARTIFICIAL SEQUENCE
  Primer
gggctcgagagtgaattctaccagtgccatacccgggaattcgtt

SEQ ID NO: 26 - DNA, 47 bp, ARTIFICIAL SEQUENCE
  Primer
ggggcggccgctatggcactggtagaattcacttctaggtttaaacg

SEQ ID NO: 27 - DNA, 46 bp, ARTIFICIAL SEQUENCE
  Primer
gggctcgagtcatacagctagataaccaaagacccgggaattcgtt

SEQ ID NO: 28 - DNA, 48 bp, ARTIFICIAL SEQUENCE
  Primer
ggggcggccgctctttggttatctagctgtatgatctaggtttaaacg

SEQ ID NO: 29 - DNA, 31 bp, ARTIFICIAL SEQUENCE
  Primer
cactagtgccaccatggtgagcaagggcgag

SEQ ID NO: 30 - DNA, 39 bp, ARTIFICIAL SEQUENCE
  Primer
gcgcggccgcttacttgtacagctcgtccatgccgagag

SEQ ID NO: 31 - DNA, 29 bp, ARTIFICIAL SEQUENCE
  Primer
ggaggctggcagcaatcgcaagggaacgg

SEQ ID NO: 32 - DNA, 29 bp, ARTIFICIAL SEQUENCE
  Primer
ccgttcccttgcgattgctgccagcctcc

SEQ ID NO: 33 - DNA, 6768 bp, Artificial Sequence
  pEF1X-sd-miR183F-sa-GFP
aatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactaggcttttgcaaaaagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttggtactaatacgactcactatagggagacccaagctggctaggtaagcttggtgcatcaacaagtttgtacaaaaaagcaggcttgtaaaacgacggcccagtgagcgcgcgtaatacgactcactatagggcgaattgggtaccgggcccccccctcgacgtaatctagaggatccctcgagggttgtaggacctccaggagaaggcagctgacccctctgcagggtctgcaggctggagagtgtgactcctgtcctgtgtatggcactggtagaattcactgtgaacagtctcagtcagtgaattaccgaagggccataaacagagcagagacagatccgcgagcaccttggagctcctcacccctttctgcctagacctctgtttccaggggtgccagggtacaaagacctcctctgctccttccccagagggcctgttccagtaccatctgcttggccgattttggcactagcacatttttgcttgtgtctctccgctgtgagcaatcatgtgtagtgccaatatgggaaaagcgggctgctgcggccacgttcacctcccccggcatcccagggtctgtgtgtctcactggctccctggcccatctggcttactgctgggtgaggagggtacagccctaccctggtgaacagccaggatccggaccatagtctggaccttgtgttaactgtgggaagagcgccctcctaaaaccaccctaactgcttcttcttcagcataggcttactggtctggctgctggaggcctcccaccatttttggcaatggtagaactcacaccggtaaggtaatgggacccggtggttctagacttgccaactatggtgtaagtgctgagctgctgaaggtctgcaccgtgccggaacctgccgatcaccaggaaggagaggggactcctgtctccagaccaccaggcagtggcagagggtgggcgctcgagtactaactggtacctcttcttttttttttgatatcctgcagaagcttgatatcgaattcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcggccgccaccgcggtggagctccagcttttgttccctttagtgagggttaattgcgcgcttggcgtaatcatggtcatagcacccagctttcttgtacaaagtggttgatagcttggaccgagctcggatccactagtccagtgtggtggaattctcgaggtcacccattcgaacaaaaactcatctcagaagaggatctgaatatgcataccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttc

SEQ ID NO: 34 - DNA, 6522 bp, Artificial Sequence
  pEF1X-sd-miR9-sa-GFP
aatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactaggcttttgcaaaaagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttggtactaatacgactcactatagggagacccaagctggctaggtaagcttggtgcatcaacaagtttgtacaaaaaagcaggcttgtaaaacgacggcccagtgagcgcgcgtaatacgactcactatagggcgaattgggtaccgggcccccccctcgacgtaatctagaggatccctcgacccgcttggtttcagcctagattcccgacctcagaaccgtcggcggtagcatcttttcctcgcctttctgaggtctcgtcgtgatagtgcgtgaggtcctggatcccatcttttcgcctccacaccacgcgcgtcccccatccgccgtcgagcgactcgagactacggaggtccaggcggtggccgggaggctgcgtggaagaggaggaggcggccaggaggcggggttggttgttatctttggttatctagctgtatgagtggtgtggagtcttcataaagctagataaccgaaagtaaaaataaccccatacactgcgcagagggcctggaacgctggtagtggccgagctgcaggagggagagccgtgccaggcgtgcctggaggctggagggctggggggagggggactcgtcgccttcaaaggttgctcagcccgagccacctcgcgatccccacccgcaacctgctcacctgggaagaaagcaagaagccgctctggggtcgcctctggaggcctggtcaaaatggtcgagtactaactggtacctcttcttttttttttgatatcctgcagaagcttgatatcgaattcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaataagcggccgccaccgcggtggagctccagcttttgttccctttagtgagggttaattgcgcgcttggcgtaatcatggtcatagcacccagctttcttgtacaaagtggttgatagcttggaccgagctcggatccactagtccagtgtggtggaattctcgaggtcacccattcgaacaaaaactcatctcagaagaggatctgaatatgcataccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttc

SEQ ID NO: 35 - protein, 13 aa, Musca domestica
RRLAANARERRRM

SEQ ID NO: 36 - protein, 13 aa, Mus musculus
RRLAANARERRRM

SEQ ID NO: 37 - protein, 13 aa, Mus musculus
RRLAAIARERRRM

(19) United States
(12) Patent Application Publication
(10) Pub. No.: US 2015/0051267 A1
(43) Pub. Date: Feb. 19, 2015

(54) BICISTRONIC GENE TRANSFER TOOLS FOR DELIVERY OF miRNAS AND PROTEIN CODING SEQUENCES

(71) Applicant: Purdue Research Foundation, West Lafayette, IN (US)

(72) Inventors: Donna M. Fekete, West Lafayette, IN (US); Michelle L. Stoller, West Lafayette, IN (US)

(21) Appl. No.: 14/461,553

(22) Filed: Aug. 18, 2014

Related U.S. Application Data
(60) Provisional application No. 61/866,598, filed on Aug. 16, 2013.

(51) Int. Cl. C12N 15/86

(52) U.S. Cl. CPC C12N 15/86; C12N 2740/10041; C12N 2710/16641
USPC 514/44 R; 435/320.1; 435/375

(57) ABSTRACT

Compositions and methods relating to microRNA (miRNA) technology are disclosed. In particular, microRNA (miRNA) expression vectors and methods for the treatment of sensory disorders, e.g., for the treatment of hearing loss, are described.

### GOVERNMENT RIGHTS

This invention was made with government support under DC002756 and DC011687 awarded by the National Institutes of Health. The government has certain rights in the invention.

### FIELD OF THE INVENTION

This invention relates to compositions and methods relating to microRNA (miRNA) technology. In particular, the invention relates to microRNA (miRNA) expression vectors and methods for the treatment of sensory disorders.

### BACKGROUND OF THE INVENTION

MicroRNAs (miRNAs) are a category of short (20-24 nt), non-coding RNAs that modulate levels of proteins in multicellular organisms via post-transcriptional regulation of gene expression by affecting both the stability and translation of mRNA (Bartel, D. P. et al. Cell 116 (2):281-9 (2004)). The activity of miRNAs is based on base-pairing with complementary sequences within target mRNA molecules (Bartel, D. P. et al. Cell 136(2):215-33 (2009)). While miRNAs resemble small interfering RNAs (siRNAs), miRNAs are derived from regions of RNA transcripts that fold back on themselves to form short hairpins, whereas siRNAs are derived from longer regions of double-stranded RNA (Bartel, D. P. et al. Cell 116 (2):281-9 (2004)).

microRNAs are found in a variety of chromosomal locations, including intergenic and intronic locations, and they may be transcribed as independent units or together with a host gene in coupled regulation of miRNA and protein-encoding gene (Lee, Y. et al. EMBO J. 23(20):4051-60 (2004); Mraz, M. et al. Blood 119(9):2110-3 (2012); Rodriguez, A. et al. Genome Res. 14(10A):1902-10 (2004)). Many miRNA genes originate from polycistronic units containing multiple discrete loops from which mature miRNAs are processed (Lee, Y. et al. EMBO J. 23(20):4051-60 (2004)).

miRNAs are generally transcribed by RNA polymerase II (Pol II) as part of capped and polyadenylated primary transcripts (pri-miRNAs) of several hundred nucleotides in length that can be either protein-coding or non-coding. A single pri-miRNA may contain up to six different miRNA precursors. (Lee, Y. et al. EMBO J. 23(20):4051-60 (2004)). The primary transcript is cleaved by the Drosha ribonuclease III enzyme to produce an approximately 70-nt stem-loop precursor miRNA (pre-miRNA), which is exported to the cytoplasm and further cleaved by the cytoplasmic Dicer ribonuclease to generate miRNA:miRNA duplexes of about 20-24 nucleotides (Lund, E. et al. Cold Spring Harb. Symp. Quant. Biol. 71:59-66 (2006)). One strand of the mature miRNA is incorporated into a RNA-induced silencing complex (RISC), which recognizes target mRNAs through imperfect base pairing with the miRNA and most commonly results in translational inhibition or destabilization of the target mRNA.

Since the discovery of miRNAs, research has focused on identifying conserved miRNA families and determining how these small molecules regulate a multitude of cellular processes that occur during carcinogenesis and normal cellular development. In development, subsets of miRNAs have been found to have expression patterns that are relatively specific for distinct cell types or organs. For example, sequence encoding the mice miRNAs miR-183, -96 and -182 are located within an intronic region on chromosome 6, and are transcribed as a single polycistronic pre-miRNA. This coordinated expression is restricted to hair cells (HCs) as they begin to differentiate in both mice and zebrafish, suggesting that these miRNAs participate in HC development.

One approach to explore the function of miRNAs is to either knockdown their levels or to force their overexpression in vivo or in vitro. Intracellular injection or transfection of miRNA mimics has been successful to overexpress mature miRNAs, although the elevated level of miRNA mimics is transient because they are not stably transduced. As an alternative, exogenous miRNAs can be stably expressed via vectors using two distinct transcriptional pathways. Some vectors use the RNA polymerase III (polIII) pathway via the U6 promoter to drive expression of pre-miRNA hairpins, while others use the RNA polymerase II (polII) pathway, for example to express two pre-miRNA sequences downstream of a tet-responsive PolII promoter. Morpholino-mediated knockdown of each of miR-183, -96 and -182 in zebrafish caused smaller inner ear sensory organ size and reduced the numbers of HCs two days after injection. Furthermore, overexpression of miR-96 and miR-182, by injection of double-stranded miRNA mimics (ds-miRNAs) into one-celled zebrafish, generated duplicate inner ears and produced supernumerary and ectopic inner ear HCs.

A major drawback of existing approaches for over- or under-expression of miRNAs is that cells overexpressing miRNAs cannot be easily identified, making subsequent phenotypic analysis difficult. To circumvent this problem, the delivery of miRNA elements may be combined with some type of reporter gene using IRES (internal ribosomal entry sites). Alternatively, constructs comprising both miRNAs and a reporter gene using two different promoters can be delivered to a cell. In the latter case, a polII- or polIII-based promoter controls the production of the miRNA and a polII-based promoter drives expression of the reporter gene. While the use of two promoters allows production of miRNAs and a protein-coding gene, the production of the two factors is not necessarily coordinated. Such a tenuous link between the relative levels of miRNAs and any associated reporter (such as green fluorescent protein (GFP)) could compromise the use of the latter as an estimate of the former in functional studies.

Given the potential importance of miRNAs in both normal and disregulated cellular growth and expansion, development of means for effectively expressing and monitoring these oligonucleotides, both in vitro and in vivo, is urgently needed. The present invention is directed to these and other important goals.

### SUMMARY OF THE INVENTION

As described herein, an approach for miRNA expression has been developed based on generating vectors that resemble the 38% of endogenous miRNA genomic loci where miRNAs are found within the introns of protein-coding genes. When used in this context, both miRNAs and an exogenous gene, such as a GFP reporter, selectable marker or functional protein, can be placed under the control of the same promoter, such as a polII-dependent promoter.

The miRNA expression vectors of the present invention can be generally characterized as comprising a promoter, an artificial intron that encodes one or more miRNAs, and optionally an exogenous gene encoding, e.g., a reporter, selectable marker or other functional protein. For example, described herein are miRNA expression vectors that may be used to deliver the three members of the sensory-specific miR-183 family from an artificial intron. In one embodiment, an miRNA expression vector is described wherein the exogenous gene is downstream of the artificial intron and encodes a reporter (e.g., GFP) while another encodes a fusion protein created between a transcription factor (e.g., Atoh1) and a tag (e.g., the hemagglutinin (HA) epitope), making it recognizable from the endogenous protein.

As demonstrated in the examples provided herein, in vitro analysis has shown that the miRNAs contained within the artificial introns are properly processed and can bind to their targets with specificity. When included, encoded reporters, selectable markers and other functional proteins are successfully translated and identifiable through immunofluorescence, functional assays, and other means. These results demonstrate that the miRNA expression vectors of the present invention can obtain simultaneous expression of miRNAs and proteins in a cell, which provides the opportunity for joint delivery of specific translational repressors (e.g., miRNA) and possibly transcriptional activators (e.g., transcription factors).

The present invention is also generally directed to the use of the miRNA expression vectors described herein in methods of altering expression of selected genes in a cell. Such methods may be used, for example, to target sensory cells in an effort to restore normal cellular functions or activities (e.g., transfection of hair cells for the treatment of hearing loss).

In a first specific embodiment, the present invention is directed to microRNA (miRNA) expression vectors comprising a promoter sequence and an artificial intron, wherein the promoter sequence is positioned upstream (5′) of the artificial intron, and wherein the artificial intron comprises a nucleic acid sequence encoding one or more miRNA genes.

In aspects of this embodiment, the promoter may be a RNA polymerase II-based promoter, a tissue specific promoter, an inducible promoter, or a constitutive promoter, or any relevant combination thereof. In a specific aspect, the promoter is elongation factor-1 alpha (EF1α) promoter.

In aspects of this embodiment, the one or more miRNA genes is selected from the group consisting of MIR9, MIR96, MIR182, and MIR183. Thus, the one or more miRNA genes may be one, any two, any three, or all four of MIR9, MIR96, MIR182, and MIR183.

In aspects of this embodiment, the miRNA expression vector comprising the promoter sequence and the artificial intron is a viral vector or a plasmid. In particular aspects, the viral vector may be an adenovirus, a retrovirus, an adeno-associated virus, or a herpes simplex virus. In a specific aspect, the retrovirus is a lentivirus. In particular aspects, the plasmid is Tol2.

In aspects of this embodiment, the miRNA expression vector optionally further comprises an exogenous gene, positioned either between the promoter and the artificial intron or downstream of the artificial intron. The exogenous gene encodes a reporter (e.g., GFP), a selectable marker or other functional protein (e.g., a transcription factor such as Atoh1). The reporter, selectable marker or other functional protein can optionally be a fusion protein created by adding a tag (e.g., the hemagglutinin (HA) epitope) to the reporter, selectable marker or other functional protein.

In a second embodiment, the present invention is directed to methods of altering expression of one or more genes in a cell, comprising introducing into a cell an miRNA expression vector comprising a promoter sequence and an artificial intron, wherein the promoter sequence is positioned upstream (5′) of the artificial intron, and wherein the artificial intron comprises a nucleic acid sequence encoding one or more miRNA genes, wherein the expression of said one or more genes is altered.

In aspects of this embodiment, the promoter may be a RNA polymerase II-based promoter, a tissue specific promoter, an inducible promoter, or a constitutive promoter, or any relevant combination thereof. In a specific aspect, the promoter is elongation factor-1 alpha (EF1α) promoter.

In aspects of this embodiment, the one or more miRNA genes is selected from the group consisting of MIR9, MIR96, MIR182, and MIR183. Thus, the one or more miRNA genes may be one, any two, any three, or all four of MIR9, MIR96, MIR182, and MIR183.

In aspects of this embodiment, the miRNA expression vector comprising the promoter sequence and the artificial intron is a viral vector or a plasmid. In particular aspects, the viral vector may be an adenovirus, a retrovirus, an adeno-associated virus, or a herpes simplex virus. In a specific aspect, the retrovirus is a lentivirus. In particular aspects, the plasmid is Tol2.

In aspects of this embodiment, the miRNA expression vector optionally further comprises an exogenous gene, positioned either between the promoter and the artificial intron or downstream of the artificial intron. The exogenous gene encodes a reporter (e.g., GFP), a selectable marker or other functional protein (e.g., a transcription factor such as Atoh1). The reporter, selectable marker or other functional protein can optionally be a fusion protein created by adding a tag (e.g., the hemagglutinin (HA) epitope) to the reporter, selectable marker or other functional protein.

In aspects of this embodiment, the miRNA expression vector is introduced into a cell via microinjection, electroporation, lipofectamine transfection, or viral infection/transduction.

In a third embodiment, the present invention is directed to methods of treating hearing impairment in a subject, comprising administering to a subject in need thereof an effective amount of an miRNA expression vector comprising a promoter sequence and an artificial intron, wherein the promoter sequence is positioned upstream (5′) of the artificial intron, wherein the artificial intron comprises a nucleic acid sequence encoding one or more miRNA genes, and wherein hearing impairment is reduced in the subject.

In aspects of this embodiment, the promoter may be a RNA polymerase II-based promoter, a tissue specific promoter, an inducible promoter, or a constitutive promoter, or any relevant combination thereof. In a specific aspect, the promoter is elongation factor-1 alpha (EF1α) promoter.

In aspects of this embodiment, the one or more miRNA genes is selected from the group consisting of MIR9, MIR96, MIR182, and MIR183. Thus, the one or more miRNA genes may be one, any two, any three, or all four of MIR9, MIR96, MIR182, and MIR183.

In aspects of this embodiment, the miRNA expression vector comprising the promoter sequence and the artificial intron is a viral vector or a plasmid. In particular aspects, the viral vector may be an adenovirus, a retrovirus, an adeno-associated virus, or a herpes simplex virus. In a specific aspect, the retrovirus is a lentivirus. In particular aspects, the plasmid is Tol2.

In aspects of this embodiment, the miRNA expression vector optionally further comprises an exogenous gene, positioned either between the promoter and the artificial intron or downstream of the artificial intron. The exogenous gene encodes a reporter (e.g., GFP), a selectable marker or other functional protein (e.g., a transcription factor such as Atoh1). The reporter, selectable marker or other functional protein can optionally be a fusion protein created by adding a tag (e.g., the hemagglutinin (HA) epitope) to the reporter, selectable marker or other functional protein.

In aspects of this embodiment, the miRNA expression vector is administered to the subject by intradermal, intramuscular, intravenous, subcutaneous, intraperitoneal, subretinal, intraocular, intracochlear, intralabyrinth, or trans-tympanic membrane administration.

In a fourth embodiment, the present invention is directed to methods of treating hearing impairment in a subject, comprising administering to a subject in need thereof an effective amount of an miRNA expression vector comprising a promoter sequence, an artificial intron and a nucleic acid sequence encoding a transcription factor, wherein the promoter sequence is positioned upstream (5′) of the artificial intron and the nucleic acid sequence encoding the transcription factor, wherein the artificial intron comprises a nucleic acid sequence encoding one or more miRNA genes, wherein the transcription factor promotes expression of a silenced gene, and wherein hearing impairment is treated in the subject.

In aspects of this embodiment, the promoter may be a RNA polymerase II-based promoter, a tissue specific promoter, an inducible promoter, or a constitutive promoter, or any relevant combination thereof. In a specific aspect, the promoter is elongation factor-1 alpha (EF1α) promoter.

In aspects of this embodiment, the one or more miRNA genes is selected from the group consisting of MIR9, MIR96, MIR182, and MIR183. Thus, the one or more miRNA genes may be one, any two, any three, or all four of MIR9, MIR96, MIR182, and MIR183.

In aspects of this embodiment, the miRNA expression vector comprising the promoter sequence, artificial intron and nucleic acid sequence encoding a transcription factor may be a viral vector or a plasmid. In particular aspects, the viral vector may be an adenovirus, a retrovirus, an adeno-associated virus, or a herpes simplex virus. In a specific aspect, the retrovirus is a lentivirus. In particular aspects, the plasmid is Tol2.

In aspects of this embodiment, the transcription factor is positioned either between the promoter and the artificial intron or downstream of the artificial intron. The transcription factor may be Atonal1, Pax2, Brn3.1, or ISL1.

In aspects of this embodiment, the miRNA expression vector optionally further comprises an exogenous gene, positioned downstream of the promoter, downstream of the artificial intron or downstream of the nucleic acid sequence encoding the transcription factor. The exogenous gene encodes a reporter (e.g., GFP) or a selectable marker. The reporter or selectable marker can optionally be a fusion protein created by adding a tag (e.g., the hemagglutinin (HA) epitope) to the reporter or selectable marker.

In aspects of this embodiment, the miRNA expression vector is administered to the subject by intradermal, intramuscular, intravenous, subcutaneous, intraperitoneal, subretinal, intraocular, intracochlear, intralabyrinth, or trans-tympanic membrane administration.

In a fifth embodiment, the present invention is directed to methods of altering the expression of one or more genes in a cell, comprising infecting a cell with a virus comprising a miRNA expression vector comprising a promoter sequence, an artificial intron and a nucleic acid sequence encoding a transcription factor, wherein the promoter sequence is positioned upstream (5′) of the artificial intron and the nucleic acid sequence encoding the transcription factor, wherein the artificial intron comprises a nucleic acid sequence encoding mir-183 family miRNAs, wherein the miRNA expression vector incorporates into the genome of the cell, wherein the mir-183 miRNAs are expressed and bind to targets in the cell, wherein the transcription factor is expressed and binds to targets in the cell, and wherein the expression of the one or more genes in the cell is altered.

In aspects of this embodiment, the promoter may be a RNA polymerase II-based promoter, a tissue specific promoter, an inducible promoter, or a constitutive promoter, or any relevant combination thereof. In a specific aspect, the promoter is elongation factor-1 alpha (EF1α) promoter.

In aspects of this embodiment, the mir-183 family miRNAs are one or more of miR-96, miR-182 and miR-183.

In aspects of this embodiment, the virus may be an adenovirus, a retrovirus, an adeno-associated virus, or a herpes simplex virus. In a specific aspect, the retrovirus is a lentivirus.

In aspects of this embodiment, the transcription factor is positioned either between the promoter and the artificial intron or downstream of the artificial intron. The transcription factor may be Atonal1, Pax2, Brn3.1, or ISL1.

In aspects of this embodiment, the miRNA expression vector optionally further comprises an exogenous gene, positioned downstream of the promoter, downstream of the artificial intron or downstream of the nucleic acid sequence encoding the transcription factor. The exogenous gene encodes a reporter (e.g., GFP) or a selectable marker. The reporter or selectable marker can optionally be a fusion protein created by adding a tag (e.g., the hemagglutinin (HA) epitope) to the reporter or selectable marker.

In a sixth embodiment, the present invention is directed to a cell comprising a miRNA expression vector of the present invention, such as supporting cells of the mammalian cochlea.

### BRIEF DESCRIPTION OF THE DRAWINGS

FIG. 1A-C. Bifunctional vector design and processing of transcripts. The vector consists of the EF1α promoter that will drive expression of the miR-183 family of genes from the intron designated by the splice donor (SD) and splice acceptor (SA) site, and an exon encoding Atoh1 fused to the hemagglutinin influenza epitope (HA). Once the plasmid is transcribed into RNA, endogenous enzymes present in transfected cells should recognize the SD and SA sites to release the intron containing the primary miRNA transcript (A) (miR-183 stem loop—SEQ ID NO:6; miR-96 stem loop—SEQ ID NO:2; miR-182 stem loop—SEQ ID NO:4), clip it into the three distinct pre-miRNAs, export them from the nucleus (B), and then further process them into mature miRNAs (C). As the miRNAs follow their own maturation pathway, the spliced Atoh1-HA-encoding polyA+ transcript is exported from the nucleus and processed as mRNA.

FIG. 2. Content and design of overexpression vectors. Each overexpression vector listed with its formal name, abbreviated name, and contents. Black boxes represent exons. Intron 1 and exons 1 and 2 were present within backbone prior to modification. Checkmarks indicate presence of artificial intronic flanking sequences. Empty spaces indicate a specific component is not found within that particular vector. TSS: transcription start site.

FIG. 3A-D. The Atoh1-HA fusion protein is functional and detectable by immunofluorescence. (A) Detection of Atoh1-HA fusion protein with HA.11 antibody in cells transfected with p183F-Atoh-HA (SEQ ID NO:1). Scale bar=100 microns. (B) Illustration of Atoh1 reporter construct. (C) Relative luciferase activity of cells transfected with Atoh1 reporter alone or with the indicated versions of the Atoh1-HA overexpression constructs. Luciferase activities are all referenced to cells transfected only with the reporter construct, which is set at 1.0. All constructs showed a significant increase in luminescence compared to the control except p183F-Atoh1(N162I)-HA. Each bar represents mean (±standard error) within each group. Each experiment was replicated at least three times. (D) Alignment of conserved Atoh1 segment between fly (SEQ ID NO:35), mouse (SEQ ID NO:36) and mouse (162) (SEQ ID NO:37). Highlighted is the location of the amino acid mutated to make Atoh1 non-functional while maintaining the HA tag. *p<0.05, **p<0.005, ***p<0.0001.

FIG. 4A-E. miRNAs are expressed from the 183F-Atoh1-HA vector and functional. (A) p183F-Atoh1-HA transfection leads to production of mature miR-183 family members. Untransfected HEK293T cells and cells transfected with pAtoh1-HA show no detectable expression of 183 family members; whereas miR-183, -96, and -182 are each detected in cells transfected with p183F-Atoh1-HA. U6 levels are provided as the loading control. (B) Illustration of miRNA-specific reporter plasmids. PsiCHECK-2 luciferase reporters contain 2 sites complementary to miR-96, -182, or -183 in the 3′UTR. (C-E) Knockdown of luciferase activity in reporters specific to each member of the miR-183 family. (C) Cells co-transfected with reporter containing miR-183 sites and p183F-Atoh1-HA showed a marked decrease in luciferase activity compared to wells transfected with the miR-183 reporter and pAtoh1-HA. Experiments in D and E were conducted in the same manner except with miR-96 or miR-182 complementary binding sites in the luciferase reporter. All showed significant decrease in luminescence. Each bar represents mean (±standard error) for each group. Each experiment was replicated at least three times. *p<0.05, **p<0.005, ***p<0.0001.

FIG. 5A-F. Plasmid containing miR-183 family and the GFP gene produces functional miRNAs and GFP protein in HEK293T cells. (A) Vector design of p183F-GFP. (B) Visualization of GFP in cells transfected with p183F-GFP. Scale bar=100 microns. (C) The miR-183 family is expressed from p183F-GFP in mammalian and avian cells. Cells transfected with p183F-GFP showed expression of mature miR-183, -96, and -182. Control (untransfected cells) and pGFP transfected cells exhibit no detectable miRNA. U6 levels serve as the loading control. (D-F) Luciferase activity is decreased by expression of miRNAs from p183F-GFP expressing vector. (D) Cells co-transfected with p183F-GFP and the psiCHECK-2 reporter containing sites complementary to miR-183 show a significant decrease in luciferase activity compared to cells co-transfected with the pGFP and reporter. E and F show results of experiments similar to D except the reporter contained different complementary binding sites: 96 for E and 182 for F. Each bar represents mean (±standard error) for each group. Each experiment was replicated at least three times. *p<0.05, **p<0.005, ***p<0.0001.

FIG. 6A-B. The miRNAs produced from both miRNA expression vectors bind to their targets with specificity. (A,B) Luciferase activity is decreased when vectors containing miR-9 are co-expressed with the miR-9 luciferase reporter but not when co-expressed with the miR-183 family expressing plasmids. (A) Luciferase activity of transfected cells containing the miR-9 luciferase reporter with pAtoh1-HA were compared to cells co-transfected with the miR-9 reporter and p183F-Atoh1-HA or p9-Atoh1-HA. (B) Experiments similar to those in A except control cells were co-transfected with miR-9 reporter and pGFP, while experimental cells were co-transfected with reporter and p183F-GFP or p9-GFP. *p<0.05, **p<0.005, ***p<0.0001. Bars represent mean (±standard error) for each group. Each experiment was replicated at least three times.

FIG. 7A-D. Detectable Atoh1-HA and functional miR-96 are expressed from the AD-183F-Atoh1-HA vector. (A) Illustration of AD-183F-Atoh1-HA. (B) Visualization of Atoh1-HA in HEK293T cells infected with AD-183F-Atoh1-HA using the HA.11 antibody. Scale bar=100 microns. (C) Mir-96 is expressed in cells infected with AD-183F-Atoh1-HA. Northern blot of HEK239T cells infected with the virus and probed for miR-96. There is a strong band at the size of mature miR-96. Bands migrating between 60-90 nt are found at various intensities in control cells and are thus likely to be endogenous transcripts of unknown identity. Uninfected cells exhibit no detectable miRNA. U6 levels serve as the loading control. (D) Knockdown of luciferase activity in reporters specific to miR-96. Cells infected with virus then subsequently transfected with a reporter containing miR-96 sites show a marked decrease in luminescence compared to uninfected cells transfected with the miR-96 reporter. Each bar represents mean (±standard error) for each group. **p<0.01

FIG. 8A-B. Detectable Atoh1-HA is expressed from the AD-Atoh1-HA vector. (A) Illustration of AD-Atoh1-HA. (B) Visualization of Atoh1-HA using the HA.11 antibody on HEK cells fixed after infection with AD-Atoh1-HA. Scale bar=100 microns.

FIG. 9A-D. Detectable GFP and functional miR-96 are expressed from the AD-183F-GFP virus. (A) Illustration of AD-183F-GFP. (B) Visualization of GFP in HEK293T cells infected with AD-183F-GFP. Scale bar=100 microns. (C) Mir-96 is expressed in cells infected with AD-183F-GFP. A Northern blot of small RNAs isolated from cells infected with the virus and probed for miR-96 show a band at the size of mature miR-96. HEK293T cells infected with virus expressing LacZ (AD-LacZ) exhibit no detectable mature miRNA-96, suggesting that AD-183F-GFP is responsible for miR-96 production. The bands migrating at ˜75 nt are present under both conditions as well as in uninfected cells (data not shown) and thus are considered non-specific. U6 levels serve as the loading control. (D) Knockdown of luciferase activity in reporters specific to miR-96. Cells infected with virus then subsequently transfected with a reporter containing miR-96 sites show a marked decrease in luminescence compared to uninfected cells transfected with the miR-96 reporter. Each bar represents mean (±standard error) for each group. **p<0.01

FIG. 10A-E. Detectable and functional miR-96 is expressed from the AAV-GFP-miR96 vector. (A) Illustration of AAV-GFP-miR96. (B) Visualization of GFP in HEK293T cells transfected with pAAV-GFP-96. Scale bar=100 microns. (C and D) miR-96 is expressed in cells transfected with pAAV-GFP-miR96 (C) or infected with AAV-GFP-miR96 (D). Untransfected or uninfected cells exhibit no detectable miRNA. (E) Knockdown of luciferase activity in reporters specific to miR-96. Cells co-transfected with pAAV-GFP-miR96 and a reporter containing miR-96 sites show a marked decrease in luciferase activity compared to cells transfected with the miR-96 reporter and pAAV-GFP. Each bar represents mean (±standard error) for each group. ***p<0.0001

FIG. 11A-E. Detectable GFP and functional miR-9 are expressed from the Tol2-GFP-9 vector. (A) Illustration of Tol2-GFP-9. (B) Visualization of GFP in avian (DF1) cells transfected with pGFP-9. Scale bar=100 microns. (C) Mature miR-9 is expressed from Tol2-GFP-9 in HEK 293T cells. Cells transfected with Tol2-GFP-9 show expression of mature miR-9. Tol2-GFP transfected cells exhibit no detectable mature miR-9. U6 levels serve as the loading control. (D) Knockdown of luciferase activity in reporters specific to miR-9. DF1 cells co-transfected with reporter containing two tandem miR-9 sites and Tol2-GFP-9 show a marked decrease in luciferase activity compared to wells transfected with the miR-9 reporter and Tol2-GFP. (E) The miRNA produced from Tol2-GFP-9 binds to its target with specificity. Luciferase activity is decreased when a vector containing miR-96 is co-expressed with the miR-96 luciferase reporter but not when co-expressed with the miR-9 expressing plasmid. Each bar represents mean (±standard error) for each group. Each experiment was repeated at least three times. ***p<0.0001

FIG. 12A-B. Tol2-GFP-9 electroporated organs show GFP expression and overexpression of miR-9 via in situ. (B′) GFP identifies the cells transfected with the Tol2-GFP-9 in the anterior cristae. No noticeable GFP expression is seen in the contralateral control ear (B). (A-A′) Sister sections labeled for miR-9 using in situ hybridization. Areas expressing GFP in B′ show corresponding increase in miR-9 expression in A′. The contralateral ear (left) is flipped to mimic the orientation of the experimental (right) ear. Scale bar=100 microns.

### DETAILED DESCRIPTION

The present invention provides miRNA expression vectors comprising a promoter, an artificial intron that encodes one or more miRNAs, and optionally exogenous genes encoding one or more selected proteins. Delivery of the miRNA expression vectors to selected cells or groups of cells results in expression of the encoded miRNAs and proteins within the cells where desired outcomes can be achieved. Because miRNAs are active in post-translational modification of mRNA, the miRNA expression vectors can be used to alter the expression of selected genes within a cell. Further, because the miRNA expression vectors can include one or more exogenous genes, the expression vectors can also be used to achieve expression of exogenous proteins within the same cell. Thus, the miRNA expression vectors of the present invention can be used for joint delivery of specific translational repressors (e.g., miRNA) and as well as transcriptional activators (e.g., transcription factors).

miRNA Expression Vectors

The miRNA expression vectors of the present invention are characterized as comprising a promoter sequence and an artificial intron, wherein the promoter sequence is positioned upstream (5′) of the artificial intron, and wherein the artificial intron comprises a nucleic acid sequence encoding one or more miRNA genes.

Promoter Sequence

The promoter sequence may comprise any promoter useful for cell-specific expression of the gene of interest. The promoter will typically be one that is recognized and acted on by RNA polymerase II, i.e., an RNA polymerase II-based promoter. It will be appreciated that such promoters are quite numerous and varied, and that they include tissue-specific promoters, as well as inducible promoters and constitutive promoters, each of which may be used in the miRNA expression vectors of the present invention. An exemplary promoter is the human elongation factor-1 alpha (EF1α) promoter. Other acceptable promoters include the human Glial Fibrillary Acid Protein (GFAP), and the cytomegalovirus (CMV) promoter. In one illustrative example, the promoter is specific for retina delivery to the outer nuclear layer and inner nuclear layer for treatment or reduction of retinal degeneration. In another illustrative example, the promoter is specific for the inner ear (e.g., Atoh1) for delivery of an expression vector to the supporting cells of damaged ears, for example, to promote hair cell regeneration.

Artificial Intron and miRNAs

The artificial intron, also termed an “intronic cassette” herein, is a polynucleotide sequence that includes one or more miRNA genes. It will be understood that the particular miRNA genes included in the intron will vary depending on the purpose for which the miRNA expression vector is constructed. Further, the sequence of the miRNA genes may be vary in that it can include the entire sequence of a particular miRNA gene as found in vivo, or it may comprise less than the entire gene, whether shortened in the upstream or downstream regions that do not encode the miRNA or within the sequence encoding miRNA itself such that there is an additional, deletion or insert of one or more nucleotides that makes up the miRNA. Particular miRNAs that have been found to be useful and that are encompassed within the scope of the invention include, but are not limited to, miR-9, and members of the sensory-specific miR-183 family, namely Mir96, Mir182, and Mir183. Therefore, the artificial intron may comprise one or more of the mice genes MIR9, MIR96, MIR182, and MIR183 (Table 1). In particular, the miRNA genes may be one, any two, any three, or all four of MIR9, MIR96, MIR182, and MIR183. The seed region, nucleotides 2-7 found at the 5′ end of the mature miRNA, is responsible for conveying target specificity for each specific miRNA. As the seed region sequence is conserved between species, mouse miRNA sequences will control the same targets in humans as in mice.

TABLE 1
Mouse miRNA sequences

| | |
|---|---|
| Mature mouse miR-96 sequence | UUUGGCACUAGCACAUUUUUGCU (SEQ ID |
| | NO: 1) |
| Pre-miRNA mouse miR-96 sequence | CCAGUACCAUCUGCUUGGCCGAUUUUGGCAC |
| | UAGCACAUUUUUGCUUGUGUCUCUCCGCUGU |
| | GAGCAAUCAUGUGUAGUGCCAAUAUGGGAAA |
| | AGCGGGCUGCUGC (SEQ ID NO: 2) |
| Mature mouse miR-182 sequence | UUUGGCAAUGGUAGAACUCACACCG (SEQ ID |
| | NO: 3) |
| Pre-miRNA mouse miR-182 sequence | ACCAUUUUUGGCAAUGGUAGAACUCACACCG |
| | GUAAGGUAAUGGGACCCGGUGGUUCUAGACU |
| | UGCCAACUAUGGU (SEQ ID NO: 4) |
| Mature mouse miR-183 sequence | UAUGGCACUGGUAGAAUUCACU (SEQ ID NO: 5) |
| Pre-miRNA mouse 183 sequence | CUGUGUAUGGCACUGGUAGAAUUCACUGUGA |
| | ACAGUCUCAGUCAGUGAAUUACCGAAGGGCC |
| | AUAAACAG (SEQ ID NO: 6) |
| Mature mouse miR-9 sequence | UCUUUGGUUAUCUAGCUGUAUGA (SEQ ID |
| | NO: 7) |
| Pre-miRNA mouse miR-9 sequence | CGGGGUUGGUUGUUAUCUUUGGUUAUCUAGC |
| | UGUAUGAGUGGUGUGGAGUCUUCAUAAAGCU |
| | AGAUAACCGAAAGUAAAAAUAACCCCA (SEQ |
| | ID NO: 8) |

The development and functional testing of intronic cassettes are described below for use in delivery of a small family of miRNAs, i.e., the miR-183 family, that are specifically expressed in primary sensory cells in a variety of vertebrate sensory systems, including vision, hearing, taste, olfaction and somatosensory systems. The evolutionarily conserved miR-183 family of miRNAs has three members (miR-183, -96 and -182) that are transcribed as a single polycistronic pri-miRNA. These miRNAs are thought to play a role in the specification of mechanosensory cells of the inner ear.

With regard to miR-96 in particular, MIR96 is an miRNA locus shown to be associated with hearing loss. For example, MIR96 has been linked to the DFNA50 locus in two families with dominant non-syndromic progressive hearing loss. Each family has a point mutation in the seed region of MIR96, but at different nucleotides. A third deafness allele of DFN50 maps to a location in the pre-miR-96 transcript that likely interferes with miRNA processing. Further supporting the link between deafness and mutations in miR-96, a semidominant deaf mouse mutant (diminuendo) has been found with yet a third point mutation in the seed region. The physiological and anatomical defects present in either heterozygous or homozygous diminuendo mice indicates that hair cells (HC) fail to fully mature.

The miR-183 family has been shown to play a role in proper HC development and maintenance in humans and animals. As described herein, the miRNA expression vectors, e.g., encoding the miR-183 family of miRNA genes, are useful therapeutic agents for treating deafness due to HC loss. The vast majority (90%) of hearing loss is categorized as sensorineural, of which the most common type results from the destruction or malformation of the HCs occupying the organ of Corti, while sparing the associated supporting cells. In embodiments of the invention described herein, the HC-promoting transcription factor, Atonal1 (Atoh1), is delivered to the supporting cells of damaged ears. Initiation and maturation of HCs require a complex regulatory network to turn off and on certain genes, and the reprogramming of supporting cells into HCs can be enhanced by combining the delivery of an activating factor (Atoh1) and repressive elements (the miR-183 family). Typically, every miR-183 family member is present during HC formation. Thus, as herein described, an miRNA expression vector was developed for efficient and simultaneous delivery of all three miRNAs along with a known HC-specification gene (Atoh1) to the same target cell population.

Exogenous Genes

As indicated above, the miRNA expression vectors may be engineered to also comprise one or more exogenous genes. While the identity of the exogenous genes that may be included in the vectors are unlimited, relevant examples include exogenous genes that encode a reporter protein that may be used to confirm that the vectors made into cells and that the genes are being successfully produced within the cell. Examples include a gene encoding GFP (green fluorescent protein), mCherry, yellow fluorescent protein (YFP), red fluorescent protein (RFP), and the like. Other useful genes that may be included are those encoding a selectable marker such as LNGFR (truncated human low affinity nerve growth factor receptor). Also included are genes encoding protein with a particular function or activity, such as a transcription factor. Examples of suitable transcription factors include Atoh1, Pax2, Brn3.1, and ISL1. Other proteins include IGF1. miRNA expression vectors encoding both miRNAs and transcription factors can serve to deliver both specific translational repressors (e.g., miRNA) and as well as transcriptional activators (e.g., transcription factors). Since many cellular processes require the joint activation and repression of downstream pathways, this delivery system provides an opportunity to achieve that dual manipulation efficiently.

It should be understood that a combination of exogenous genes can be included in one miRNA expression vector, such as genes encoding both a reporter and a functional protein, such as a transcription factor.

In some applications use of vectors encoding reporter proteins, selectable markers or other functional proteins that bear a tag, such as the hemagglutinin (HA) epitope or histidine tag will be useful and it should be understood that the exogenous genes of the present invention may thus encode such proteins that have been fused to a peptide tag.

The exogenous gene may be under the control of the same promoter as the artificial intron or a different promoter. In preferred aspect, a single promoter directs transcription of both the miRNAs encoded by the artificial intron as well as the exogenous gene. The exogenous gene may be located between the promoter and the artificial intron, or it may be located downstream of the artificial intron, which in turn is downstream of the promoter.

Vectors

As will be understood, the miRNA expression vectors of the present invention comprise the promoter sequence, artificial intron and optional exogenous gene in the context of a larger polynucleotide sequence. Thus, the promoter sequence, artificial intron and optional exogenous gene can be considered a genetic cassette that can be inserted into particular vectors or plasmids that are suitable for delivering the genetic cassette to a particular cell, cell type or group of cells. The vectors and plasmids will vary depending on the ultimate destination of the genetic cassette. However, both viral vectors and plasmids have been found to be effective means of delivery. In particular aspects, the viral vector may be, but is not limited to, an adenovirus, a retrovirus (such as a lentivirus), an adeno-associated virus, or a herpes simplex virus. In particular aspects, the Tol2 plasmid may be used.

Means for Introducing miRNA Expression Vectors into Cells

Means for introducing the miRNA expression vectors of the present invention into cells will be understood by one of ordinary skill in the art. Acceptable means will vary depending on the type of vector used in the construction to the miRNA expression vectors (e.g., viral or plasmid), the cell type, and the location of the cells. For in vitro applications, the miRNA expression vectors are typically introduced to cells via microinjection, electroporation or lipofectamine transfection, or viral infection/transduction

For in vivo applications, the miRNA expression vectors are typically prepared in a pharmaceutically acceptable formulation and then administered to a subject via parenteral means, such as intradermal, intramuscular, intravenous, subcutaneous, intraperitoneal, subretinal, intraocular, intracochlear, intralabyrinth, or trans-tympanic membrane administration. Suitable means include needle (including microneedle) injectors, needle-free injectors and infusion techniques. A pharmaceutically-acceptable formulation is one that is suitable for administration to an animal, such as a human or other mammal. It will typically be one that does not include any animal-sourced material (ASM-free) when the subject is a human. Suitable examples include miRNA expression vectors diluted in water-for-injection, or 0.9% saline.

Methods of Using the miRNA Expression Vectors

The miRNA expression vectors of the present invention are extremely flexible and they can be used in a variety of applications, whether in vitro, ex vivo and in vivo. For example, they can be used as research tools to study the activity of particular miRNAs and/or proteins encoded by the exogenous genes in the laboratory setting, and as means for delivering and expressing miRNAs and/or proteins in a subject. Such expression in a subject can be used in the treatment of a disease, disorder or condition.

Thus in one embodiment, the present invention includes methods of expressing miRNA molecules in a cell. The method comprises introducing into a cell an miRNA expression vector of the present invention, wherein the artificial intron comprises nucleic acid encoding one or more miRNA genes, and wherein said miRNA molecules are expressed.

The present invention also includes methods of altering expression of one or more genes in a cell. The method comprises introducing into a cell one or more of the miRNA expression vectors of the present invention. Expression of the one or more miRNAs and the exogenous gene, when present, can alter the apparent expression of targeted genes in the cell. For example, the miRNAs can be directed to bind mRNA corresponding to a particular protein with the result being a decrease in the amounts of the protein in the cell, thus altering expression of the targeted gene. Alternatively, or in addition, the exogenous gene can encode a transcription factor, for example, that induces expression of a targeted gene (such as of a silenced gene), thus again altering expression of the targeted gene. Altering expression thus refers to both increases and decreases in gene expression.

In a related embodiment, the invention includes methods of altering the expression of one or more genes in a cell. The method comprises infecting a cell with a virus comprising a miRNA expression vector comprising a promoter sequence, an artificial intron and a nucleic acid sequence encoding a transcription factor. The promoter sequence is positioned upstream (5′) of the artificial intron in the miRNA expression vector and the nucleic acid sequence encoding the transcription factor. In this embodiment, the artificial intron comprises a nucleic acid sequence encoding mir-183 family miRNAs. Upon infection of the cell, the miRNA expression vector incorporates into the genome of the cell, wherein the mir-183 miRNAs are expressed and bind to targets in the cell, wherein the transcription factor is expressed and binds to targets in the cell. Such action by the miRNAs and the transcription factor results in altering of the expression of one or more genes in the cell. In a particular aspect of this embodiment, the transcription factor is Atoh1 and the cells are supporting cells of the mammalian cochlea. The mir-183 family miRNAs are one or more of miR-96, miR-182 and miR-183. In aspects of this embodiment, the virus may be an adenovirus, a retrovirus, an adeno-associated virus, or a herpes simplex virus. In a specific aspect, the retrovirus is a lentivirus.

In aspects of this embodiment, the miRNA expression vector optionally further comprises an exogenous gene, positioned downstream of the promoter, downstream of the artificial intron or downstream of the nucleic acid sequence encoding the transcription factor. The exogenous gene encodes a reporter (e.g., GFP) or a selectable marker. The reporter or selectable marker can optionally be a fusion protein created by adding a tag (e.g., the hemagglutinin (HA) epitope) to the reporter or selectable marker.

Because many miRNAs have been identified as having involvement in the development of sensory organs in a subject, methods of treating a sensory disorder in a subject are encompassed within the scope of the invention. Such methods comprise identifying a subject having a sensory disorder and administering to the subject an effective amount of a microRNA (miRNA) expression vector of the present invention. The miRNA expression vector comprises a promoter upstream of an artificial intron, wherein the artificial intron encodes one or more miRNA genes. In one aspect, the miRNA gene is a member of the sensory-specific miR-183 family, which comprises miR-96, miR-182 and miR-183. In one aspect, the vector further comprises a reporter gene downstream of the artificial intron. In an alternative aspect, the vector comprises an artificial intron downstream of a reporter gene.

The sensory disorder may be, but is not limited to, one selected from the group consisting of a vision, hearing, taste, olfaction or somatosensory disorder. In particular, the sensory disorder is a hearing disorder, e.g., hearing loss or impairment.

In a specific embodiment the present invention includes methods of treating hearing impairment in a subject. The methods comprise administering to a subject in need thereof an effective amount of a miRNA expression vector of the present invention. As described above, expression of the one or more miRNAs and the exogenous gene, when present, can alter the expression of targeted genes in the cell, for example, promote expression of a silenced gene.

The one or more miRNAs encoded by the expression vector are selected from miR-9, miR-96, miR-182 and miR-183. In one aspect, the one or more miRNAs encoded by the expression vector are miR-96, miR-182 and miR-183. In another aspect, the one or more miRNAs encoded by the expression vector are each of miR-9, miR-96, miR-182 and miR-183.

The exogenous gene encodes Atonal1, Pax2, Brn3.1, or ISL1.

The cells comprising the targeted genes are preferably hair cells of the inner ear, and supporting cells of the mammalian cochlea.

These methods result in a reduction of the hearing impairment in the subject, such as a reduction of at least about 5%, 10%, 15%, 20%, 25%, 30%, 35%, 40%, 45%, 50%, 55%, 60%, 65%, 70%, 75%, 80%, 85%, 90%, 95% or even more.

In a related embodiment, the invention is directed to methods of treating hearing impairment in a subject, comprising administering to a subject in need thereof an effective amount of an miRNA expression vector comprising a promoter sequence, an artificial intron and a nucleic acid sequence encoding a transcription factor, wherein the promoter sequence is positioned upstream (5′) of the artificial intron and the nucleic acid sequence encoding the transcription factor, wherein the artificial intron comprises a nucleic acid sequence encoding one or more miRNA genes, wherein the transcription factor promotes expression of a silenced gene, and wherein hearing impairment is treated in the subject.

The one or more miRNAs encoded by the expression vector are selected from miR-9, miR-96, miR-182 and miR-183. In one aspect, the one or more miRNAs encoded by the expression vector are miR-96, miR-182 and miR-183. In another aspect, the one or more miRNAs encoded by the expression vector are each of miR-9, miR-96, miR-182 and miR-183.

The exogenous gene encodes Atonal1, Pax2, Brn3.1, or ISL1.

The silenced gene may be, but is not limited to, Sox2, Hes1, or ISL1.

Hearing impairment is treated in the subject such that restoration of at least about 5%, 10%, 15%, 20%, 25%, 30%, 35%, 40%, 45%, 50%, 55%, 60%, 65%, 70%, 75%, 80%, 85%, 90%, 95% or even more of the subjects hearing is achieved.

In a specific embodiment, the invention is directed to methods of treating hearing impairment in a subject, comprising administering to a subject in need thereof an effective amount of an miRNA expression vector comprising a promoter sequence, an artificial intron and a nucleic acid sequence encoding a transcription factor, wherein the promoter sequence is positioned upstream (5′) of the artificial intron and the nucleic acid sequence encoding the transcription factor, wherein the artificial intron comprises a nucleic acid sequence encoding miR-96, miR-182 and miR-183, wherein the transcription factor is Atonal1, and wherein hearing impairment is treated in the subject.

In each of the embodiments and aspects described herein, the expression of one or more target genes may be down-regulated or up-regulated by the miRNAs encoded by the miRNA expression vectors of the invention. The miRNAs may target one or more different transcripts in a cell. There are large numbers of potential targets for each miRNA, and new targets are being discovered as time passes. It will be understood that the present invention is not limited to the regulation of specific targets, but includes regulation of the many different targets depending on the identity of the particular miRNAs encoded by the miRNA expression vector. The publication by Groves et al. (Annu. Rev. Neurosci. 2013. 36:361-81) provides a recent review of some targets, reproduced in part in Table 2, for miRNA 183 family members.

TABLE 2
Partial List of Targets for miRNA 183 family members.
miR-96 target (model organism)

ACVR2B (h)
ADCY6 (h)
AQP5 (m, h)
ARRDC3 (m)
AVIL (m)
CACNB4 (h)
CASP2 (m)
CELSR2 (m, h)
COL2A1 (h)
FMNL2 (h)
FN1 (h)
FOXO1 (h)
FOXO3 (h)
GPC3 (h)
HTR1B (h)
KRAS (h)
LMX1A (h)
MITF (h)
MYLK (h)
MYO1B (h)
MYRIP (m, h)
NEUROD4 (m)
NR3C1 (m)
ODF2 (m, h)
PGR (h, rhesus, not m)
RAD51 (h)
REV1 (h)
RYK (m, h)
SEMA6D (h)
SLC19A2 (h)
SLC39A1 (h)
SLC39A3 (h)
SLC39A7 (h)
SPAST (h)
ZIC1 (h)

miR-182 target (model organism)

ACTR2 (h)
ADCY6 (h)
ARRDC3 (m)
BCL2 (h)
BRCA1 (h)
CASP2 (m)
CCND2 (h)
CLOCK (h)
CREB3L1 (h)
EPAS1 (h)
FOXF2 (h)
FOXO1 (h)
FOXO3 (h)
MET (h)
MITF (h)
MTSS1 (h)
MYO1C (h)
MYRIP (h)
NCALD (h)
PCDH8 (h)
RAB3GAP2 (h)
RASA1 (h)
RGS17 (h)
RNF212 (h)
SLC30A1 (h)
SLC30A7 (h)
SLC35A5 (h)
SLC39A1 (h)
SLC39A7 (h)
SOX2 (h)
SPAST (h)
TBX1 (m)

miR-183 target (model organism)

EGR1 (h)
ITGB1 (h)
KIF2A (h)
PDCD4 (h)

Abbreviations:
h, human;
m, mouse

In each of the embodiments and aspects described herein, the transcription factor may bind to genomic DNA in the cell at one or more targets and down-regulate expression of one or more genes. Alternatively, the transcription factor may bind to genomic DNA in the cell at one or more targets and up-regulate expression of one or more genes. The miRNAs and the transcription factor may target the same genes. The miRNAs and the transcription factor may target different genes.

As discussed above, the miRNA expression vectors defined herein are may function as dual-delivery vectors that express a traceable transcription factor and one or more miRNA genes. In one illustrative example, the vectors express an entire miRNA family, using endogenous sequences, contained within a single artificial intron. In another illustrative example, a transcription factor (e.g., Atoh1) fused to the reporter gene (e.g., GFP) is produced from the vector and is transcriptionally active. In another illustrative example, the transcription factor Atoh1 fused to a tag (e.g., an HA tag) is produced from the vector and is transcriptionally active. In one example the target cells (e.g., HCs) receiving the bifunctional vector can be monitored via the reporter gene or tag.

As described herein, the miRNA expression vectors and methods may be used for treatment of sensory disorders in both human clinical medicine and animal veterinary medicine. Thus, the patient treated using the methods herein described can be human or can be a laboratory, agricultural, domestic, or wild animal. Thus, the methods described herein are useful for treating humans, laboratory animals, e.g., rodents (such as mice, rats, rabbits, etc.) monkeys, chimpanzees, domestic animals (e.g., dogs and cats), agricultural animals and wild animals in captivity.

In one illustrative example, the expression vector described herein uses a polII-based promoter to control the expression of an entire miRNA family within the context of an artificial intron and its downstream reporter gene (e.g., GFP). In another example, co-expressed miRNA family members are overexpressed in a coordinated manner with one another for healthy cell function. For example, in one illustrative example, an expression vector was designed to express a functional transcription factor (in the place of GFP) and the miR-183 family, while still maintaining the ability to monitor transfected cells by creating the Atoh1-HA fusion protein. The expression vectors as herein described allow delivery of the miRNA genes into tissues that are difficult to access or to transfect (e.g., the mouse organ of Corti depleted of HCs). Thus, the miRNA expression vectors and methods described herein are useful for multiple overexpression uses to study a variety of different complex cellular systems and for various therapeutic purposes, including, for example, hair cell regeneration.

While certain embodiments of the present invention have been described and/or exemplified herein, it is contemplated that considerable variation and modification thereof are possible. Accordingly, the present invention is not limited to the particular embodiments described and/or exemplified herein.

### EXAMPLES

### Example 1

### Bifunctional Plasmid Construction

The Atoh1 coding sequence was PCR amplified from the pEF1-Atoh-IRES-GFP vector. To facilitate cloning and protein detection, one primer introduced an EcoRI site, while the other primer added an influenza hemagglutinin (HA) tag (YPYDVPDYA; SEQ ID NO:12) to the C-terminus of Atoh1 coding sequence and a NotI site (the sequences of all the primers used are provided in Tables 3-6). Atoh-HA was cloned into pEF1X (a modified version of the Invitrogen pEF1/myc-His C vector where the Neomycin cassette was removed; provided by Cliff Ragsdale) as an EcoRI-NotI fragment and the entire fusion was verified by sequencing (Purdue Genomics Center).

To construct an artificial miR183-containing intron, a SalI-HindIII fragment containing a splice donor, three restriction sites XbaI, BamHI and XhoI, polypyrimidine tract, branch point, and a splice acceptor was generated by PCR (the tract, branch point and splice acceptor sequences were taken from Lin and Ying, 2004; primers are provided in Table 3) and cloned into pME-MCS (Kwan et al., 2007), generating pMCS-SDA. PCR primers were generated based on published sequences. PCR amplification was used to extract primary miRNA DNA that comprises all three members of the miR-183 family from the mouse genome, and to flank the genomic DNA with SpeI and SalI (primers provided in Tables 3 and 4). This sequence was inserted between the XbaI and XhoI sites found within the intron contained within pME-MCS-sda to create pME-MCS-sd-miR183F-sa. Kpn1 was used to extract the artificial intron containing about 800 bp of mouse miR-183 family genomic primary miRNA sequence from pME-MCS-sd-miR183F-sa. The KpnI site was used to insert the intron with the miR-183 family upstream of the Atoh1-HA fusion protein in pEF1X to generate pEF1X-sd-miR183F-sa-Atoh-HA.

TABLE 3
Primer sequences used to construct miR-183 family/Atoh1-HA bifunctional cassette.

| Splice | | |
|---|---|---|
| Donor/Acceptor | Sequence | Location |
| Forward | gcggtcgacgtaatctagaggatccctcgagtactaactggtacctcttc | |
| | (SEQ ID NO: 13) | |
| Reverse | gcaagcttctgcaggatatcaaaaaaaaaagaagaggtaccagttagtactc | |
| | (SEQ ID NO: 14) | |
| miR-183 Family | | |
| 183-96 Forward | gcactagtggttgtaggacctccagga (SEQ ID NO: 15) | Chr6: 30169792- |
| | | 30169810 |
| 183-96 Reverse | tccagactatggtccggatcctggctgttcaccagggtagggctg (SEQ ID | Chr6: 30169333- |
| | NO: 16) | 30169357 |
| 182 Forward | cctggtgaacagccaggatccggaccatagtctggaccttgtgtt (SEQ ID | Chr6: 30166079- |
| | NO: 17) | 30166102 |
| 182 Reverse | gcctcgagcgcccaccctctgccactg (SEQ ID NO: 18) | Chr6: 30165809- |
| | | 30165828 |
| Atoh1-HA | | |
| Forward | cgaattcgccaccatgtcccgcctgctgcatgcagaag (SEQ ID NO: 19) | |
| Reverse | cgcgcggccgcctaagcgtaatctggaacatcgtatgggtaactggcctcatcaga | |
| | gtcactgtaatg (SEQ ID NO: 20) | |

TABLE 4
Primers containing sequences complementary to miRNA of interest for creation of
miRNA reporters.
miR96 reporter

| | |
|---|---|
| Forward | gggctcgagagcaaaaatgtgctagtgccaaacccgggaattcgtt (SEQ ID NO: 21) |
| Reverse | ggggcggccgctttggcactagcacatttttgcttctaggtttaaacg (SEQ ID NO: 22) |

miR182 reporter

| | |
|---|---|
| Forward | gggctcgagcggtctgagttctaccattgccaaacccgggaattcgtt (SEQ ID NO: 23) |
| Reverse | ggggcggccgctttggcaatggtagaactcacaccgtctaggtttaaacg (SEQ ID NO: 24) |

miR183 reporter

| | |
|---|---|
| Forward | gggctcgagagtgaattctaccagtgccatacccgggaattcgtt (SEQ ID NO: 25) |
| Reverse | ggggcggccgctatggcactggtagaattcacttctaggtttaaacg (SEQ ID NO: 26) |

miR9 reporter

| | |
|---|---|
| Forward | gggctcgagtcatacagctagataaccaaagacccgggaattcgtt (SEQ ID NO: 27) |
| Reverse | ggggcggccgctctttggttatctagctgtatgatctaggtttaaacg (SEQ ID NO: 28) |

pEF1X-sd-miR183F-sa-GFP vector was constructed by extracting GFP from pAAV2.1-CMV-eGFP3-WPRE (Karali et al., 2011; provided by Alberto Auricchio) via PCR using the primers shown in Table 5. Using the SpeI and NotI sites found on the 5′ and 3′ ends respectively, GFP was inserted downstream of the miRNA-containing artificial intron in pMCS-sd-miR183F-sa to create pMCS-sd-miR183F-sa-GFP. The pEF1x vector was converted to a Gateway Destination vector (Invitrogen) by inserting cassette B from the Gateway Conversion Kit (Invitrogen) to create pEF1X-cB. A LR recombination reaction between pMCS-miR183F-GFP and pEF1x-B generated pEF1X-sd-miR183F-sa-GFP (SEQ ID NO:33).

TABLE 5
Primers used to amplify GFP from pAAV2.1-CMV-
eGFP3-WPRE.

| GFP | |
|---|---|
| Primers | |
| Forward | cactagtgccaccatggtgagcaagggcgag |
| | (SEQ ID NO: 29) |
| Reverse | gcgcggccgcttacttgtacagctcgtccatgccgagag |
| | (SEQ ID NO: 30) |

pEF1X-sd-miR9-sa-Atoh1-HA and pEF1X-sd-miR9-sa-GFP (SEQ ID NO:34) were constructed in a similar manner as the aforementioned vectors except miR-9 genomic sequence was inserted into the artificial intron instead of the miR-183 family. PCR was used to extract the endogenous mouse miR-9-1 sequence and flanking regions using primer sequences described (Shibata et al., 2008).

For each miRNA reporter, primers were designed to contain two sequences that were fully complementary to the mature miRNA of interest (miR-183, -96, -182, or -9). These sites were separated by a 17 nucleotide spacer sequence. For all cases, the forward primer contained a XhoI site, while the reverse primer housed a NotI site to allow the resulting PCR fragments to be introduced downstream of the Renilla luciferase gene located in the psiCHECK-2 vector (Promega).

### Example 2

### Mutation of Atoh1-HA Fusion Protein

To introduce the N162I substitution in Atoh-1, site-directed mutagenesis was performed with Quikchange 2XL (Strategene) according to the manufacturer's instructions. Primers (Table 6) were designed to induce a point mutation to change the amino acid 162 from Asparagine to Isoleucine in the Atoh1-HA fusion protein encoded by pEF1X-sd-miR183F-sa-Atoh-HA creating pEF1X-sd-183F-sa-Atoh1(N162I)-HA.

TABLE 6
Sequences used to introduce Atoh1 mutation.

| Atoh1 | |
|---|---|
| Mutation | |
| Primers | |
| Forward | ggaggctggcagcaatcgcaagggaacgg |
| | (SEQ ID NO: 31) |
| Reverse | ccgttcccttgcgattgctgccagcctcc |
| | (SEQ ID NO: 32) |

### Example 3

### HEK293T Plasmid Transfection

HEK293T cells were cultured with modified DMEM supplemented with L-glutamine, antibiotics, and 10% calf serum. Using Lipofectamine 2000 (Invitrogen), cells seeded in 6-well plates were transfected with plasmids of interest. Collection time was assay dependent.

### Example 4

### HEK293T Immunostain and Imaging

Cells transfected with pEF1X-sd-miR183F-sa-Atoh1-HA or pEF1X-sd-miR183F-sa-GFP were fixed 24 hours post-transfection with 4% paraformaldehyde. The following primary antibodies (1:1000) were used: for detection of the HA tag, anti-HA.11 mouse IgG₁ monoclonal (Covance); for detection of GFP, anti-GFP rabbit polyclonal (Molecular Probes). Secondary antibodies (1:500) used were Alexa Fluor (Molecular Probes) 488 anti-mouse IgG₁ and Alexa Fluor 488 anti-rabbit IgG. Immunostained cells were imaged under the Nikon E800 fluorescence microscope with the 20× objective.

### Example 5

### Atoh1 and miRNA Luciferase Assays

Relevant to Examples below, luciferase assays were conducted on cells 24 hours after transfection, where the cells were lysed and luciferase activity was assessed using the dual luciferase assay kit (Promega) in the Luminoskan Ascent luminometer (Thermo Electron). For the Atoh1 luciferase assays, the firefly luciferase luminescence readings (at 560 nm) were normalized to the Renilla luciferase readout (at 480 nm) to account for variation in transfection efficiency. In the case of the miRNA luciferase assays the ratio was inverted: the Renilla luciferase readout was normalized to the firefly luciferase readout. These ratios are expressed as relative luciferase activity. Experimental values were referenced to the control values which were arbitrarily set to one. Each treatment condition was conducted at least in duplicate. The experiments were repeated at least three times.

### Example 6

### Northern Blots

HEK 293T or DF1 cells seeded in 35 mm plates were lysed ˜30 hours post-transfection and small RNAs were collected according to manufacturer's instructions using the PureLink miRNA Isolation Kit (Invitrogen). Small RNA (300 ng) was probed for miR-183, -96, or -182 using the High Sensitive miRNA Northern Blot Assay Kit (Signosis), a chemiluminescence system, according to manufacturer's instructions.

### Example 7

### Statistical Analysis

All results are reported as mean±standard error. The mean of each group is computed from measurements collected from at least three independent experiments. Statistical significance was determined by using a one-way analysis of variance with block (ANOVA), which was followed by Tukey's or Tukey-Kramer's multiple comparisons test (SAS 9.3). P-values below 0.05 were considered statistically significant.

### Example 8

### Construction of Bifunctional Atoh1-HA and miRNA Expression Vector

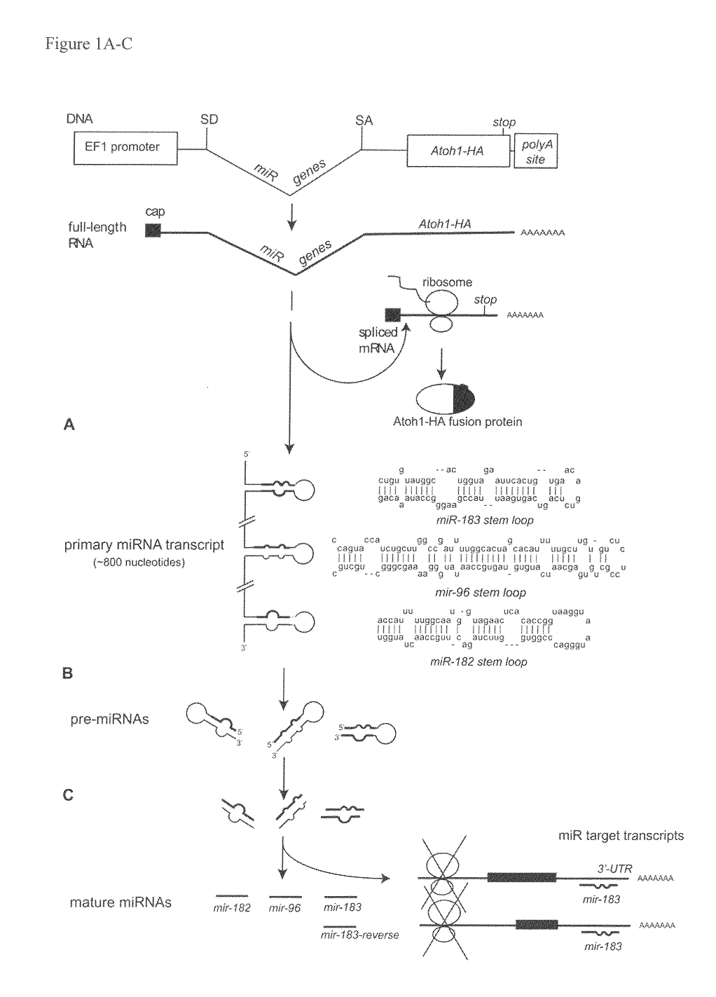

In order to coordinate the expression of the miRNAs and Atoh1 with high precision within the same cell, both elements were synthesized from the same RNA transcript. An artificial intron containing the miRNAs was placed downstream of EF1α (human elongation factor 1 alpha; pEF1X) and upstream of Atoh1 coding sequence (FIG. 1). Within the mouse genome, about 3.5 kb of sequence separates Mir182 from the nearest other family member Mir96. To accommodate the size restrictions of certain delivery vectors, such as the RCAS avian retrovirus and adeno-associated virus, the large intervening stretch between Mir182 and Mir96 was removed while retaining the natural pre-miRNA sequences for all 3 family members. Thus, all of the endogenous sequence between Mir183 and Mir96 (˜120 bp) along with ˜100 bp of sequence flanking the end of each pre-miRNA sequence was kept. Then, the pre-miR-182 sequence, with 120 bps flanking each end, was fused to the Mir183/Mir96 fragment by PCR.

The combined pri-miRNA sequences were inserted into the artificial intron sequence. This artificial intron of only ˜100 bps contains a splice donor site at the 5′ end of the pri-miRNAs. The 3′ end flanking the pri-miRNAs houses a branch point domain, polypyrimidine tract, and splice acceptor site. The polypyrimidine tract allows spliceosome assembly, while the branch point is necessary for the cell to recognize that a splicing event should occur to excise the element between the splice donor and acceptor sites.

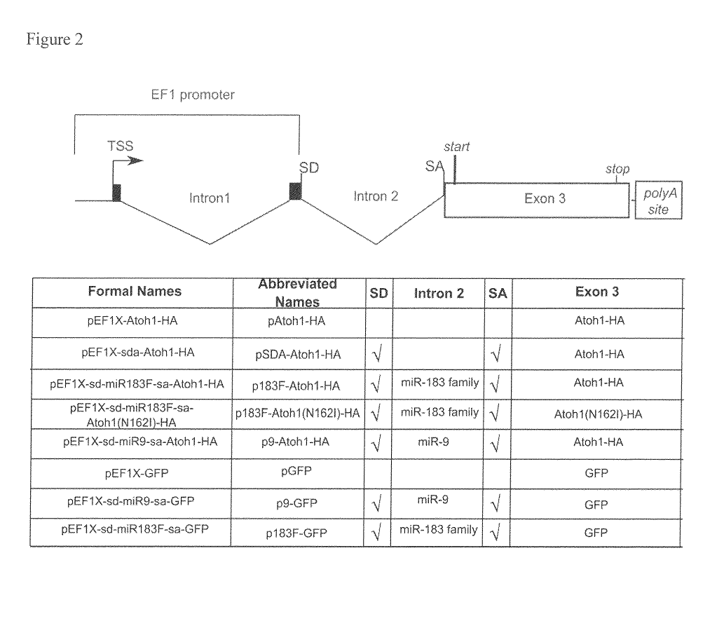

Downstream of the miRNA intron is the Atoh1 coding region. This Atoh1 sequence was proven bioactive by its ability to induce ectopic HCs in utero. To facilitate the detection of Atoh1 expression from transfected plasmids, an influenza hemagglutinin (HA) peptide tag (YPYDVPDYA) was fused in-frame to the Atoh1 coding sequence. FIG. 2 displays the overall design of the resulting plasmid, pEF1X-sd-miR183F-sa-Atoh1-HA, hereafter referred to as p183F-Atoh-HA1 (SEQ ID NO:1, where nucleotides 1-4 and 793-845 correspond to synthetic intron, nucleotides 5-792 correspond to mouse miRNA183 family sequence, and nucleotides 855-1940 correspond to murine Atoh1 sequence with hemagglutinin epitope fusion). FIG. 2 also provides details for the introns and exons of the other constructs and their abbreviated names that will be introduced below.

### Example 9

### Confirmation of Atoh1-HA Production and Function from a Bifunctional Cassette

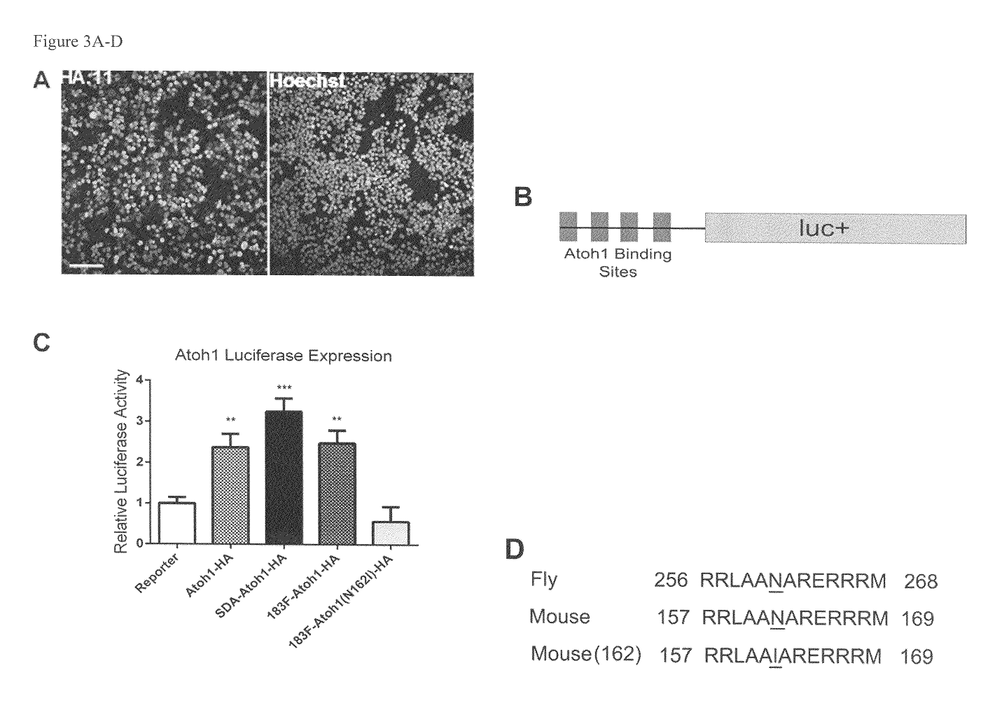

To ascertain that Atoh1 is expressed from this bicistronic system, HEK293T cells transfected with p183F-Atoh1-HA were stained with anti-HA antibody. In cells 24 hours after transfection, HA-positive staining was readily seen in the nuclei using immunofluorescence (FIG. 3A), consistent with the fact that Atoh1 is a transcription factor. No HA-positive staining was seen in mock-transfected cells, demonstrating that the signal in p183F-Atoh1-HA-transfected cells is specific (data not shown).

While the immunofluorescence suggests that HA-tagged Atoh1 was expressed and properly localized, it remains possible that the addition of a peptide hinders its bioactivity. To ensure that the HA-tagged Atoh1 was functional, its ability to activate the expression of a luciferase-based reporter gene (4E-box), which has a firefly (FF) luciferase coding sequence placed under the control of four Atoh1-binding sites, was tested. In addition, hpRL-SV40 (Promega), a plasmid with Renilla luciferase driven by a constitutive promoter, was included for normalization. HEK293T cells transfected with pAtoh1-HA showed a 138% increase (p=0.0031) in FF luminescence, compared to those transfected with the pEF1X empty vector. Similarly, cells transfected with pSDA-Atoh1-HA and p183F-Atoh1-HA showed significant increase in FF luciferase luminescence (FIG. 3C; pSDA-Atoh1-HA, 225% increase, p<0.0001: p183F-Atoh1-HA, 149% increase, p=0.0004).

To ensure that this increase in FF luciferase expression required a functional Atoh1 protein, a highly conserved asparagine in the homeobox domain was mutated to disrupt Atoh1 function. p183F-Atoh1(N162I)-HA was generated, which expresses, Atoh1-HA with the N162I substitution (this mutation is analogous to the point mutation affecting amino acid 261 in the fly) (FIG. 3D). In cells transfected with p183F-Atoh1(N162I)-HA, mutant Atoh1-HA was still detectable by immunofluorescence (data not shown), although its ability to activate FF luciferase expression was diminished (69% decrease in luminescence relative to 3 vectors carrying the wild type Atoh1 sequence; ANOVA; p<0.0001). The N162I mutation seems to act as a dominant negative, as the luminescence in p183F-Atoh1(N162I)-HA transfected cells decreased by 43% compared to the control (FIG. 3C; p=0.8213). Atoh1 is believed to act as a heterodimer that binds to other bHLH (basic helix loop helix) transcription factors such as E47. Expression of N162I likely prevents the formation of functional Atoh1 heterodimers by depleting the pool of endogenous E47 or other such transcription factors. Results showed clearly that functional HA-tagged Atoh1 is expressed from these constructs.

### Example 10

### Confirmation of miRNA Production and Function from a Bifunctional Cassette

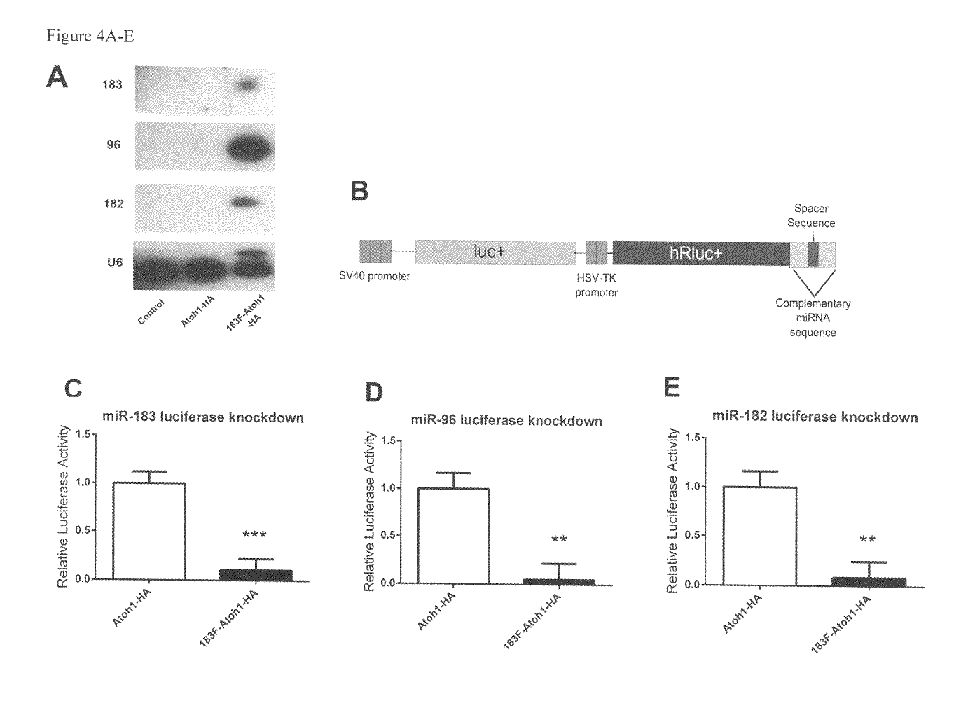

To assess whether the miRNAs were synthesized from the artificial intron, small RNAs collected from HEK293T cells 30 hours after p183F-Atoh1-HA transfection were analyzed by Northern blots. While none was detected in untransfected or pAtoh1-HA transfected cells, bands corresponding to mature miRNA of each 183 family member were seen in p183F-Atoh1-HA-transfected cells (FIG. 4A). It is notable that the relative levels of the three miRNAs are distinctly different, with miR-96 most prominent. The observation that these family members are not uniformly expressed has also been reported for murine retina and cochlea.

A dual luciferase assay system was used to confirm bioactivity of the miR-183 family miRNAs produced from the cassette. For each miRNA, a reporter construct was created beginning with psiCHECK-2 (Promega), into which two binding sites complementary to a mature miRNA and separated by a spacer sequence were inserted downstream of the Renilla luciferase gene (FIG. 4B). The psiCHECK-2 vector also contains the firefly luciferase gene driven off a separate promoter, so that luminescence from the firefly protein serves as an internal transfection control. Reporters containing the miRNA binding sites for miR-182, miR-96 or miR-183 were co-transfected with p183F-Atoh1-HA into HEK293T cells. The luminescence ratio (corrected for transfection efficiency) from the experimental wells was compared to control wells transfected with the relevant miR-183 family reporter and the pAtoh1-HA plasmid lacking the miRNA intron. As shown in FIG. 4C-E, each miRNA-reporter construct showed a significant knockdown in luminescence compared to its corresponding control (miR-96: 95% knockdown, p=0.0013; miR-182: 92% knockdown, p=0.0008; miR-183: 89% knockdown, p<0.0001). Thus, all 3 miRNAs are produced from the bifunctional cassette and appear functional.

### Example 11

### Overexpression of Functional miRNAs from GFP Expression Vectors

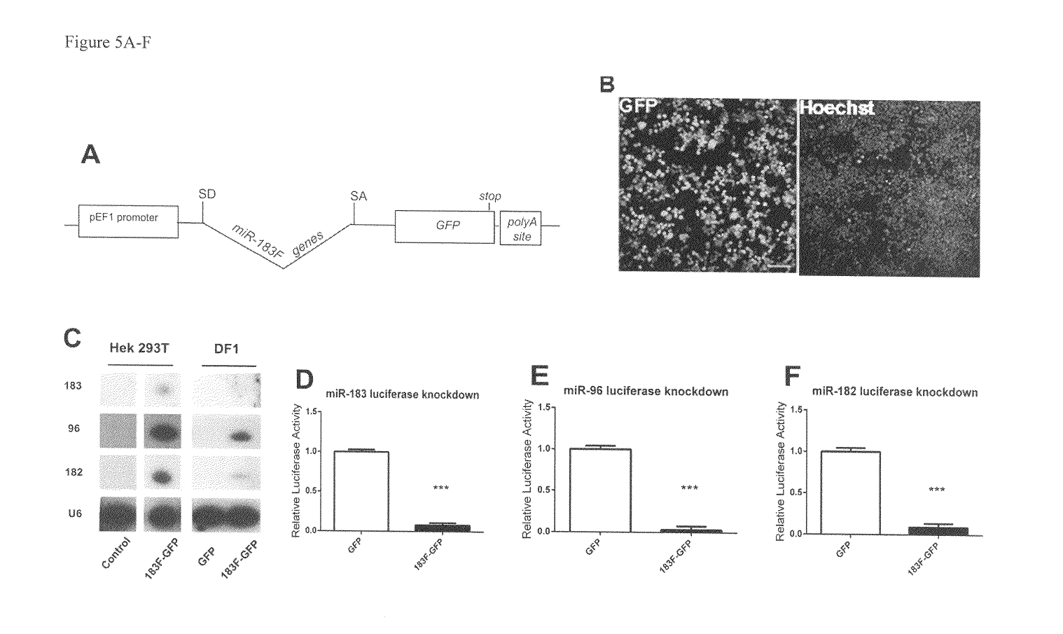

The miR-183 family was expressed alone to assess how much of an impact this family can have on HC development by itself. The Atoh1-HA coding region was replaced with GFP, which would allow the identification of cells expressing transfected miRNA constructs. Furthermore, as the design of p183F-GFP is the same as the p183F-Atoh1-HA (FIG. 5A), phenotypic analysis using vectors with or without Atoh1-HA is less likely to be confounded by changes in the processing of the RNA transcripts that may affect transcript levels.

To test whether functional GFP protein is expressed from p183F-GFP, HEK293T cells were transfected and observed not only for direct GFP fluorescence but also after enhancing the signal with anti-GFP antibodies. After 24 hours, green emissions were detected from the majority of fixed, transfected cells both before (not shown) and after immunofluorescence (FIG. 5B).

All three mature miRNAs of the miR-183 family could be detected in HEK293T cells transfected with p183F-GFP but not in cells transfected with a GFP vector lacking the miRNA intron, as assessed by Northern blots (FIG. 5C). Notably, miRNA expression levels appear to remain consistent regardless of the identity of downstream coding sequence (FIG. 5C; HEK293T cells).

To ascertain whether avian cells are able to process and express mammalian miRNAs, small RNAs from DF1 cells (chicken embryo fibroblast cells) transfected with p183F-GFP or pGFP were analyzed by Northern blotting. While both p183F-GFP and pGFP transfected groups expressed GFP fluorescence (data not shown), only those transfected with p183F-GFP showed bands corresponding to miR-182, -96 and -183 (FIG. 5C; DF1 cells). The relative levels of the miR-183 family levels appeared lower in transfected DF-1 cells than HEK293T cells. This discrepancy could result from the species difference of the transfected cells (chicken vs. human, respectively) or the difference in their respective tissue origins (embryonic day 10 fibroblasts versus fetal kidney, respectively). Nevertheless, data demonstrated clearly that the miRNAs from p183F-GFP can be processed and produced in avian cells, allowing the option of using them in avian-specific vectors, like RCAS.

Using the miRNA luciferase reporters discussed above, the function of the three miRNAs expressed from p183F-GFP was tested. Compared to HEK293T cells transfected with pGFP (which lacks the miRNA-producing intron), p183F-GFP transfection showed significant decrease in the expression of all three targets (miR-96: 97% knockdown p<0.0001; miR-182: 91% knockdown, p<0.0001; miR-183: 92% knockdown, p<0.0001) (FIG. 5D-F). These observations demonstrated that corresponding miRNA produced from the intron can successfully knockdown its specific target.

### Example 12

### miRNAs Produced from Expression Vectors Bind with Specificity

To demonstrate the specificity of the knockdown mediated by these intronic miRNAs, another luciferase-based reporter was generated with two sites complementary to miR-9, a miRNA unrelated to the miR-183 family. Two intronic-miR-9 expression vectors with different downstream protein coding sequences (Atoh1-HA or GFP) were also generated to ensure this miR-9 reporter functions properly.

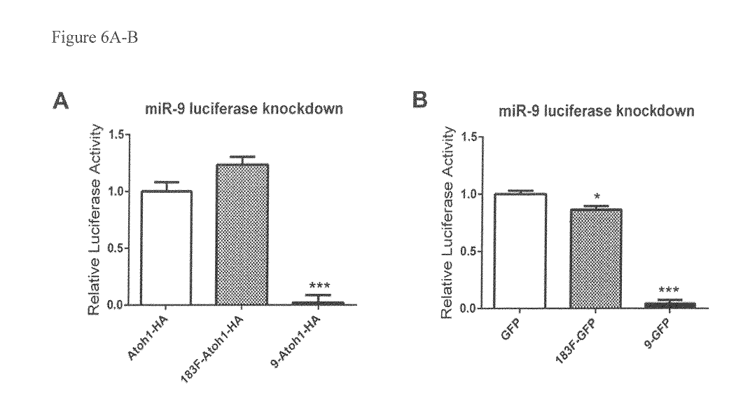

In cells transfected with miR-9 reporter, co-transfection of p9-Atoh1-HA or p9-GFP resulted in greater than 95% decrease in Renilla luciferase expression (98% decrease, p<0.0001 for p9-Atoh1-HA; 96% decrease, p<0.0001 for p9-GFP), demonstrating that these miR-9 expression vectors are functional. The comparable knockdown with both suggests that miR-9 vectors are similar to the 183F-expressing plasmid series in being effectively processed from the artificial intron regardless of the identity of downstream coding sequences. Co-transfection of p183F-Atoh1-HA or p183F-GFP, while capable of knocking down the expression of 183F-based reporters (see above), showed only negligible effects on the Renilla luciferase level from the miR9 reporter (23% increase, p=0.10 for p183F-Atoh1-HA; 13% decrease, p=0.02 for p183F-GFP) (FIG. 6). These data suggest that intronic miRNAs produced from 183F- and miR-9-expressing vectors regulate the expression of their target genes with high specificity.

### Example 13

### Viral Expression Vectors

The plasmids described in Examples 1-12 are appropriate for short-term expression of the bifunctional cassettes comprising miRNAs and one or more protein coding sequences. The following examples discuss additional delivery vectors that were constructed for use in long-term expression of the miRNAs and proteins.

For in vivo delivery of the bifunctional cassettes, such as within the mammalian inner ear, two different viruses were utilized, namely adenovirus (AD) and adeno-associated virus (AAV). Each virus shows preferential transduction of specific cochlear cells types. AD has a double-stranded genome. Its larger size allows for the insertion of large constructs that cannot fit in other size-limited vectors like the AAV, but it does not integrate into the genome so mutations caused by random integrations are not a problem. This virus can infect dividing and non-dividing cells, as well as elicit high-levels of gene expression (Lai et al., 2002). In addition, an AD vector has successfully transduced supporting cells of the mammalian cochlea (Izumikawa et al., 2005; Sheffield et al., 2011; Staecker et al., 2001), making it a good candidate for promoting hair cell regeneration from supporting cells.

AAV is an attractive gene delivery vehicle for long-term expression as it does not induce a humoral immune reaction (Lai et al., 2002). The wild-type single-stranded AAV genome consistently integrates into chromosome 19 in the human genome (Lai et al., 2002). This consistency minimizes concern regarding random insertions of the transgene into the endogenous genome that could potential cause mutations. In regards to its applications within the ear, it has been shown to efficiently transduce hair cells (Kilpatrick et al., 2011), which may be advantageous for attempts to deliver survival factors to rescue damaged hair cells. Certain serotypes can transduce supporting cells (Stone et al., 2005). However, overall, AAVs appear to be far better at transducing hair cells, at least in vivo in postnatal cochleas (Kilpatrick et al., 2011). While many advantages exist for using AAV, its smaller genome limits the size of the insert that it can contain to approximately 4.0 kb, and its single-stranded genome results in slower production of the transgene in comparison to AD. However, both vectors are used, with AD primarily used for hair cell regeneration, and AAV primarily used for hair cell repair/rescue.

### 13.1 Adenovirus (AD) Construction

The bifunctional cassette containing the miR-183 family and Atoh1-HA was placed in the adenoviral vector AD5-CMV-V5-DEST (Invitrogen) through a LR Clonase reaction with pME-MCS-sd-183F-sa-Atoh1-HA and AD5-CMV-V5-DEST creating AD-sd-183F-sa-Atoh1-HA. Alternate versions of the vector containing only the miR-183 family-intron or Atoh1-HA were also generated. AD-sd-183F-sa-GFP and AD-Atoh1-HA were created by a LR Clonase II reaction between pME-MCS-sd-183F-sa-GFP or pME-MCS-Atoh1-HA and AD5-CMV-V5-DEST.

### 13.2 Adenovirus (AD) Production

Adenoviral stocks were produced according to the manufacturer's instructions (ViralPower Adenoviral Gateway Expression Kit, Invitrogen). Briefly, HEK293A cells were seeded at 5×10⁵ cells per well. The following day, when the cells were 90-95% confluent, growth media was removed from the cells and replaced with transfection media. The cells were then transfected with 1 μg of viral vector, previously digested with Pac I, using 6 μL of Lipofectamine. After an overnight incubation, the transfection media was removed and replaced with growth media. Two days post-transfection, the cells were transferred to a 10-cm plate and media was replaced until a noticeable cytopathic effect (CPE) was observed. Once the cells reached 80% CPE, the cells and supernatant were removed by gently spraying the bottom of plate with media. This mixture was transferred to a 15 mL conical tube and was subjected to multiple freeze-thaw cycles (−80° C. for 30 minutes followed by 15 minutes at 37° C.). After the third and final cycle, the mix was centrifuged at 3000 rpm for 15 minutes. The supernatant was removed and stored at −80° C. Amplification and titering of the stock were performed as directed by the manufacturer's protocol. Titers of amplified stocks ranged from 7×10⁸ to 9×10⁸ pfu/ml (plaque forming units/ml) (Table 7).

TABLE 7
Adenoviruses produced and titers.

| Virus | Abbreviations | Titer (pfu/mL) |
|---|---|---|
| AD-sd-183F-sa-Atoh1-HA | AD-183F-Atoh1-HA | 8 × 10⁸ |
| AD-sd-183F-sa-GFP | AD-183F-GFP | 9 × 10⁸ |
| AD-Atoh1-HA | AD-Atoh1-HA | 7 × 10⁷ |
| Ad-CMV/V5-GW/lacZ | AD-LacZ | 8 × 10⁸ |

### 13.3 HEK293T Immunostaining and Imaging

HEK293T cells were infected at a multiplicity of infection (MOI) of 3 with the appropriate adenovirus. After 24 hours, cells were fixed with 4% paraformaldehyde in PBS for 10 minutes at room temperature. Following 1× PBS rinses, 10% goat serum (Vector Laboratories) with 0.05% Triton X-100 (Sigma-Aldrich) was used to block against non-specific binding for 1 hour at room temperature. The appropriate primary antibody was diluted in the blocking solution, added to the cells, and allowed to incubate overnight at 4° C. The following primary antibodies (1:1000) were used: for detection of the HA tag, anti-HA.11 mouse IgG1 monoclonal (Covance, Indianapolis, Ind., USA); for detection of GFP, anti-GFP rabbit polyclonal (Molecular Probes, Eugene, Oreg., USA). The following day the cells were rinsed with 1× PBS and incubated for 15 minutes at room temperature with the appropriate secondary antibody (1:500) diluted in 1× PBS. The secondary antibodies used were Alexa Fluor (Molecular Probes) 488 anti-mouse IgG1 and Alexa Fluor 488 anti-rabbit IgG. Then cells were counterstained using Hoescht (1:10000) in 1× PBS. Immunostained cells were imaged under the E800 fluorescence microscope (Nikon) with the 20× objective.

### 13.4 Adenovirus Luciferase Assays

The production and activity of miRNAs from the adenoviruses were also tested using the luciferase assay described in Stoller et al., 2013, but with some modifications. HEK293T cells were seeded in 6-well plates 16-24 hours prior to infection and maintained in HEK293T growth media with 10% FBS (Atlantic Biologicals) instead of 10% Calf Serum (Gibco). Prior to infection, the growth media was removed and replaced with transfection media. Then, cells were infected at a MOI of 3 with the relevant adenovirus. After six hours, cells were subsequently transfected with a miRNA reporter (psiCHECK-2; 0.02 ug) and a carrier plasmid (pME-MCS; 0.98 ug) using 2.4 uL of Lipofectamine per well. The cells were allowed to incubate overnight. The following morning, the transfection media was removed and replaced with HEK293T media containing 10% FBS. 24 hours after transfection, the cells and media were removed from the wells and placed in conical tubes. The cell-media mixture was spun at 90 g for 5 minutes. The supernatant was removed and replaced with 1× PBS to remove any residual media. The tubes underwent a second spin at 90 g for 5 minutes. After removing the PBS, 500 uL of 1× PLS was added to the conical tubes to lyse the cells. The subsequent analysis of the samples was performed in a similar manner as discussed in Example 5.

### 13.5 Construction of Adeno-Associated Virus (AAV) for miR-96 Expression

To study the effect of overexpressing wildtype miR-96 in diminuendo mice, an AAV vector containing the pre-miR96 sequences was constructed. The primary miR-96 sequence was extracted from pME-MCS-sd-183F-sa using primers flanked with XhoI sites. These sites allowed insertion of the pre-miR96 sequence into the intron of pME-MCS-sda (discussed in Stoller et al., 2013) to create pME-MCS-sd-96-sa. To allow for easy insertion of the intronic pre-miR96 sequence into pAAV2.1-CMV-eGFP3-WPRE, the vector was made Gateway compatible by placing a Gateway Conversion Cassette between GFP and the bGH sequence of the vector creating pAAV2.1-CMV-eGFP3-cB-WPRE. A LR Clonase II reaction between pME-MCS-sd-96-sa and pAAV2.1-CMV-eGFP3-cB-WPRE yielded an AAV vector capable of overexpressing miR-96, pAAV2.1-CMV-eGFP3-sd-96-sa-WPRE (SEQ ID NO:10). Abbreviations are listed in Table 8.

TABLE 8
Abbreviations for Adeno-Associated Viruses.

| Plasmid | Abbreviations |
|---|---|
| pAAV2.1-CMV-eGFP3-WPRE | pAAV-GFP |
| pAAV2.1-CMV-eGFP3-sd-96-sa-WPRE | pAAV-GFP-96 |

### 13.6 Northern Blots

HEK 293T seeded in 35 mm plates were lysed 30 to 48 hours post-transfection (2 μg) or infection (AD: 3 MOI; AAV: 50000 MOI) and small RNAs were collected according to manufacturer's instructions using the PureLink miRNA Isolation Kit (Invitrogen). Small RNA (300 ng to 2 μg) was prepared for electrophoresis by adding the appropriate volume of Novex TBE-Urea sample buffer (2×; Invitrogen) and handled according to manufacturer's instructions. Then, the samples are probed for miR-96 using the High Sensitive miRNA Northern Blot Assay Kit (Signosis), a chemiluminescence system, excluding the provided gel loading buffer. The blot was performed according to manufacturer's instructions except only 1.5-2 μL of the provided ladder was loaded. Membranes were exposed to ECL Hyperfilm (GE) for 1-15 minutes depending on the samples being assessed.

### 13.7 AAV Luciferase Assays

Testing of the expression and production of miRNAs from the AAV vectors was conducted in a similar manner as in Stoller et al., 2013 (discussed above in Example 5). These experimental values were referenced to the control values (wells co-transfected with pEF1X, the appropriate psi-CHECK2 miRNA reporter, and pAAV-GFP) which were arbitrarily set to one.

### 13.8 Pixel Quantification

Pixel measurements were performed on one of the three series of 16 micron sections for each embryo injected with either virus. Images from HCS1 immunostained samples were collected on the E800 fluorescent scope (Nikon) and converted to 16-bit images using ImageJ. After thresholding, each image had the same size area measured for pixel values. This area encompassed the entire medial and lateral portions of the dorsal region of the saccule in order to account for normal and possible abnormal locations of hair cells. Pixels were totaled across the entire saccule (4-6 sections). The HCS-Immunoreactivity values for the injected (right) saccule were compared to the uninjected (left) saccule values.

### 13.9 Statistics

All results are reported as mean±standard error. The mean of each group is computed from measurements collected from at least two independent experiments. Statistical significance from in vitro experiments was determined by using a one-way analysis of variance with block (ANOVA), which was followed by Tukey's or Tukey-Kramer's multiple comparisons test (SAS 9.3). In vivo quantification used paired t-tests (GraphPad Software). P-values below 0.05 were considered statistically significant

### Example 14

### Replication-Defective Adenovirus (AD5) Containing the Bifunctional Cassette Produces High Titers and Detectable Levels of miR-96 and Atoh1-HA or GFP In Vitro

Previous research showed that adenoviruses can successfully transduce supporting cells during murine cochlear development in utero (Sheffield et al., 2011), and the supporting cells of 3-month old and 1-month old mouse cochleas in vivo (Staecker et al., 2001). More importantly, this viral serotype was previously used to deliver Atoh1 to supporting cells within a damaged sensory epithelium (Izumikawa et al., 2005). While this strategy was able to elicit hair cell generation when applied 4 days after an ototoxic assault, application of the virus 7 days after the assault was not able to spur hair cell generation (Izumikawa et al., 2008). Thus, generating hair cells after long-term hair cell loss must require additional factors. These factors may be the hair cell-expressed miR-183 family that could assist Atoh1 to promote hair cell formation, possibly by repressing factors that either maintain the supporting cell fate or actively inhibit the hair cell phenotype.

An adenovirus was constructed and produced containing the bifunctional cassette containing the miR-183 family and Atoh1-HA using AD5-CMV-V5-DEST (Invitrogen) as the backbone (see Examples 13.1 and 13.2 above). This virus and the control virus AD5-CMV-V5-GW-lacZ were propagated and amplified using the ViraPower Gateway Expression Kit (Invitrogen). Both viruses yielded the same number of plaque forming units (pfu) per mL (8.0×10⁸ pfu/mL). After a virion infects a HEK293T cell, the virus begins to replicate and package new virions. Eventually, the number of virions will fill the entire cell, causing it to burst. The lysing of the cell and subsequent infection and bursting of the cells surrounding it will create a visible plaque (a cell-sparse region) on the plate. The number of viral plaques per unit volume of media indicates the number of functional viral particles, which is a more relevant titer than measuring the number of viral genomes per ml, because so many virions are not infectious. Since the control and AD-183F-Atoh1-HA stocks have the same titers, this indicates that the presence of the cassette does not seem to impair viral reproduction.

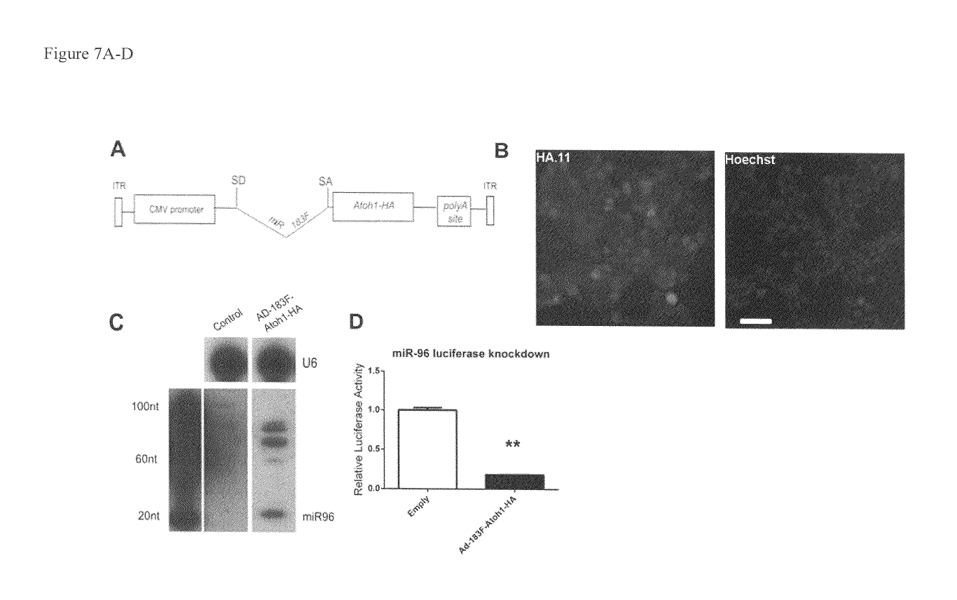

To determine whether cells infected with the adenovirus produce the Atoh1-HA protein, HEK 293T cells were infected at 3 MOI. Then, they were harvested 24 hours later and stained for the HA epitope using the HA.11 antibody. Noticeable HA levels were detected in infected cells (FIG. 7B) while uninfected cells showed no staining (data not shown). In similarly infected cells, the production of at least one of the mature miRNAs was verified by harvesting small RNAs 30 hours after infection. These samples were compared to uninfected HEK293T cells using Northern blots. FIG. 7C shows detectable expression of mature miR-96 in cells infected with the virus while the uninfected cells show none. It also appeared that the miR-96 produced is functional based on an altered luciferase protocol designed to give the virus time to produce mature miR-96. At least six hours prior to transfecting HEK293T cells with miR-96 luciferase reporter, the cells were infected with the virus. One day after transfection, the cells were harvested. The luminescence ratios of these samples were compared to wells transfected with the reporter but uninfected by virus. Cells infected with the AD-183F-Atoh1-HA virus showed a significant decrease in luminescence compared to the controls (82% knockdown; n=3; p=0.0013) (FIG. 7D).

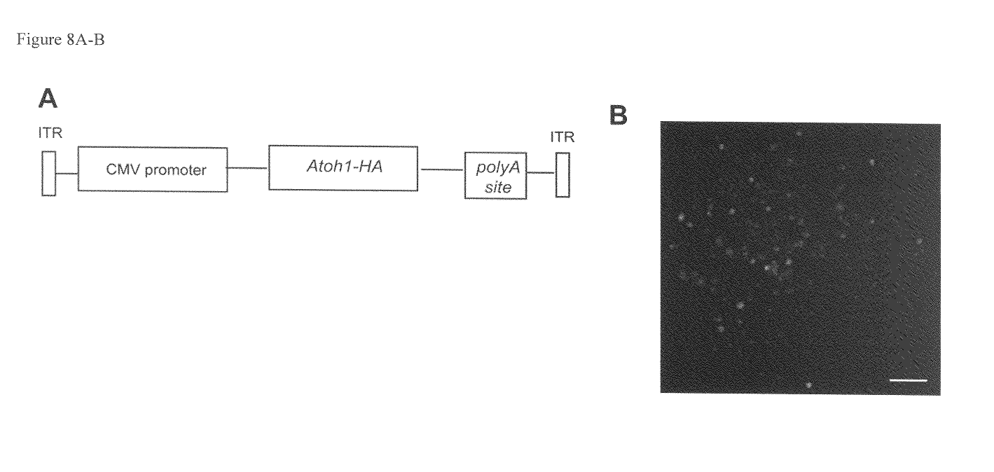

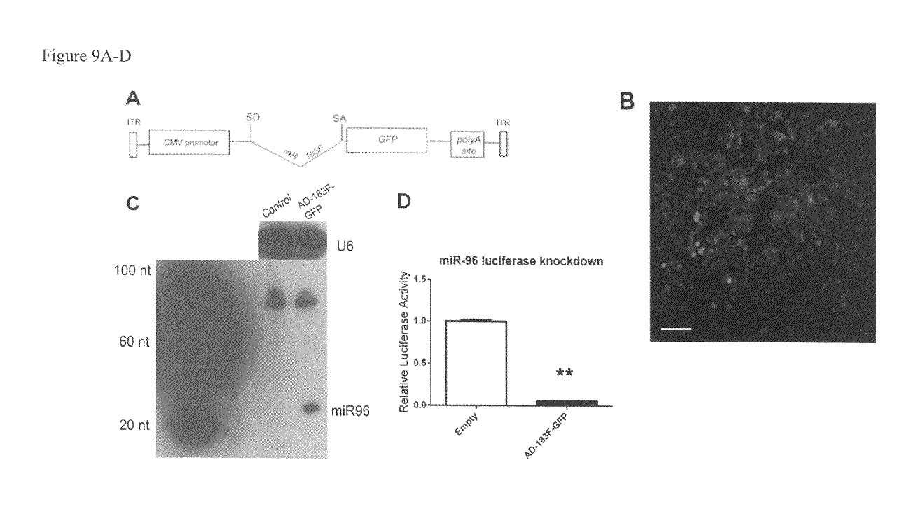

Along with using the virus to treat sensory hair cell loss in vivo, the virus may have applications in vitro for directing the specification or differentiation of stem cells. Embryonic or induced pluripotent stem cells can be coaxed to produce hair-cell-like cells. These cells have the appearance of immature hair cells, but fewer than 1% of stem cells typically convert to this cell type (Oshima et al., 2010). It is possible that the percentage of hair cells generated from these stem cells may be increased by delivering additional factors such as the miR-183 family and Atoh1. In order to provide the proper controls for this experiment and the in vivo studies, a virus was generated that expresses the miR-183 family and GFP (AD-183F-GFP) as well as a second virus to only express Atoh1-HA (AD-Atoh1-HA). Each virus was propagated using the same method described above. FIGS. 8 and 9 show that the reporter elements present in each virus (HA and GFP) can be detected in infected cells.

To validate the expression of the miRNAs from AD-183F-GFP, Northern blots probing for mature miR-96 were conducted on HEK293T cells infected with virus. These blots clearly display miR-96 production (FIG. 9C). To ensure that viral infection per se does not increase endogenous miR-96 expression, cells infected with AD-lacZ were harvested and probed for mature miR-96. No detectable miR-96 was present in these samples (FIG. 9C). These results suggest that the miRNA sequence in AD-183F-GFP is responsible for producing the miR-96. Luciferase assays confirm that the miR-96 is functional (94% knockdown; n=3; p=0.0003) (FIG. 9D).

### Example 15

### Replication-Defective Adenovirus (AAV) Containing miR-96 and GFP Produces Mature miRNA and GFP In Vitro

An AAV vector was constructed that overexpresses wild-type miR-96. The AAV virus, more specifically an AAV serotype 8 virus, was chosen due to its ability to transduce inner hair cells and outer hair cells well (Kilpatrick et al., 2011). The diminuendo mouse contains an A>T mutation in the Mir96 gene that will cause an A>U change in the seed region of mature miR-96 (Lewis et al., 2009). In these mice, the hair cells fail to fully mature into functioning inner hair cells and outer hair cells, which results in a loss of hearing (Kuhn et al., 2011). This phenotype could be due to the mutant miRNA failing to regulate its normal targets. Thus, overexpression of the wild-type miRNA in hair cells may be able to rescue them.

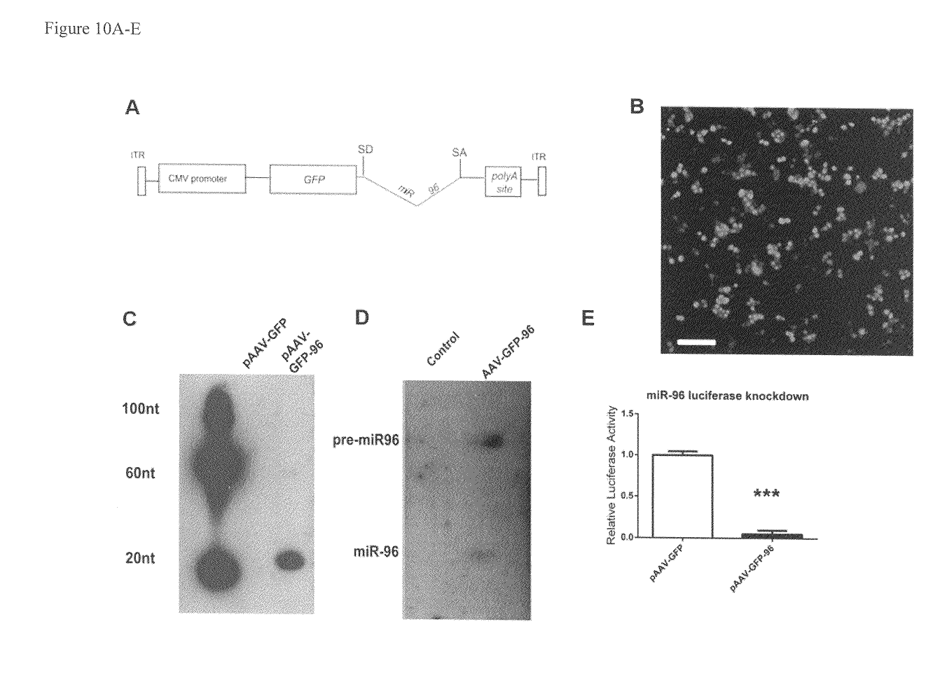

After creating AAV-GFP-miR96-WPRE (AAV-GFP-miR96), the function of the miR-96 intron was tested in vitro, using the same miRNA bioactivity assays discussed previously. As a control, viral plasmid lacking the miR-96 intron (AAV-GFP) was co-transfected with psicheck96 and compared to that of cells transfected with AAV-GFP-miR96 and psicheck96. There was a 95% knockdown of luciferase activity (FIG. 10E; n=9; p<0.0001). Northern blots of HEK293T transfected (10C) or infected (10D) with AAV-GFP-miR96 also seemed to confirm that mature miR-96 is produced from vectors in both plasmid and viral forms.

### Example 16

### Expression and Overexpression of miR-9 in the Avian Inner Ear

Advancements in sequencing technology have expanded the pool of miRNAs found within the inner ear. One of these miRNAs, miR-9, has many well-known functions within the brain, but little information is known about its localization and function within the inner ear. Mir-9 serves in a negative feedback loop that controls proliferation and differentiation in neural stem cells (Zhao et al., 2009) and triggers the Cajal-Retizus fate (Shibata et al., 2008). Besides progressing cell fate in the brain, miR-9 also triggers a late-progenitor cell fate in the retina (Torre et al., 2013). Although miR-9 is found primarily in the brain and eye, a number of Northern blots, in situs, and microarrays have shown that miR-9 is also found in the otocyst (Friedman et al., 2009; Shibata et al., 2008; Weston et al., 2006). So far, only the presence of this miRNA in the ear has been reported.

Using miRNA target prediction programs, the Hes1 transcript was found to be a predicted target of the miR-9 in the human, mouse, zebrafish, and chicken. This interaction was supported when Bonev and researchers (2012) showed that miR-9 repression of Hes1 transcripts initiated neuronal differentiation in precursors (Bonev et al., 2012). This finding is especially intriguing considering the role of Hes1 in hair cell development. Knockouts of Hes1 showed an increase in the number of inner hair cells along with an upregulation of Atoh1 in mice (Zine et al., 2001). Therefore, it would be beneficial to discover the precise localization of miR-9 within the ear and study what if any effects overexpression of miR-9 would have on inner ear development. The following examples identify the location of miR-9 within the avian inner ear as well as discuss the creation and testing of vectors to overexpress miR-9.

### 16.1 TOL2-GFP-miR9 Construction

The miR-183 family-intron fragment was inserted into the construct pT2K-CAGGS-EGFP (provided by Dr. Yoshiko Takahashi; (Sato et al., 2007). By co-electroporating this construct with a vector containing a transposase (pCAGGS-T2TP), the intron containing the miRNAs, which is flanked by two Tol2 sites, will be randomly integrated into the chicken genome allowing for stable overexpression of the miR-9. To allow for easy insertion and expression of different microRNAs in the future, pT2K-CAGGS-EGFP was converted to a Gateway vector using the Gateway Conversion Kit (Invitrogen). Once this was accomplished, LR Clonase II (Invitrogen) was used to transfer the miR-9 family/intron cassette from pME-MCS-sd-miR9-sa into the altered pT2K-CAGGS-EGFP backbone.

### 16.2 DF1 Plasmid Transfections

UMNSAH-DF1 cells (abbreviated DF-1 cells; ATTC #CRL-12203) were cultured using modified DMEM (Sigma-Aldrich) supplemented with 1 mM L-glutamine (Gibco), 1 mM penicillin-streptomycin (pen-strep; Gibco), 10% fetal calf serum, and 1% chicken serum (Sigma-Aldrich). Cells were seeded in 6-well plates (Costar) 20-24 hours prior to transfection. Immediately preceding transfection, growth media was removed and replaced with transfection media: 10% fetal calf serum (Atlanta Biologicals) in Optimem with Glutamax (Gibco). Plasmids of interest were transfected using Lipofectamine 2000 (Invitrogen) according to manufacture instructions.

### 16.3 DF1 Immunostain and Imaging

Cells used for immunostaining were transfected with 2 μg of plasmid by mixing 5 μL of Lipofectamine with plasmid DNA in 200 μL of Optimem with Glutamax. Once the mix was added, the transfection mixture remained on the wells for 6 hours and was subsequently removed and replaced with normal growth media. After 24 hours, cells were fixed with 4% paraformaldehyde for 10 minutes at room temperature. Following PBS rinses, 10% goat serum (Vector Laboratories) with 0.05% Triton X-100 (Sigma-Aldrich) was used to block against non-specific binding for 1 hour at room temperature. The appropriate primary antibody was diluted in the blocking solution and allowed to incubate with the cells overnight at 4° C. The following primary antibody (1:1000) was used for detection of GFP: anti-GFP rabbit polyclonal (Molecular Probes, Eugene, Oreg., USA). The following day the cells were rinsed with PBS and incubated for 15 minutes at room temperature with the appropriate secondary antibody (1:500) diluted in PBS. The secondary antibody used was Alexa Fluor 488 anti-rabbit IgG. Then cells were counterstained using Hoescht (1:10000) in PBS. Immunostained cells were imaged under the E800 fluorescence microscope (Nikon) with the 20× objective.

### 16.4 miRNA Target Validation

Testing of the expression and production of miRNAs from the Tol2 vectors was conducted in a similar manner as stated in Stoller et al., 2013 except in DF1 cells. These experimental values were referenced to the control values (wells co-transfected with pEF1X, the appropriate psi-CHECK2 miRNA reporter, and Tol2-GFP) which were arbitrarily set to one.

### 16.5 Northern Blots

HEK 293T seeded in 35 mm plates were lysed 30 hours post-transfection (of 2 μg of plasmid DNA) and small RNAs were collected according to manufacturer's instructions using the PureLink miRNA Isolation Kit (Invitrogen). Small RNA (300 ng) was prepared for electrophoresis by adding the appropriate volume of Novex TBE-Urea sample buffer (2×; Invitrogen) and handled according to manufacturer's instructions. Then, the samples are probed for miR-9 using the High Sensitive miRNA Northern Blot Assay Kit (Signosis), a chemiluminescence system, excluding the provided gel loading buffer. The blot was performed according to manufacturer's instructions except only 1.5-2 μL of the provided ladder was loaded. Membranes were exposed to ECL Hyperfilm (GE) for 1-15 minutes depending on the samples being assessed.

### 16.6 Electroporation of Tol2-GFP-9

Fertilized chicken eggs were acquired from Purdue University Farms and incubated at 37° C.-38° C. After the removal of the amnion and application of Chick Ringer's solution, the Tol2-GFP-9 plasmid and pCAGGS-T2TP were co-injected into the fluid space of the chicken otocyst on embryonic day 3 (E3), which encompassed Hamburger and Hamilton (HH) stages 16-18 (Hamburger and Hamilton, 1951), at a 2:1 molar ratio and electroporated into the prosensory primordium of either the anterior crista or the posterior cristae as described (Chang et al., 2008). Two 10-volt square wave current pulses, 50 milliseconds long and spaced 10 milliseconds apart, were administered to platinum paddle electrodes using a TSS20 Ovodyne electroporator connected to an EP21 Current Amplifier (Intracel, UK). Embryos were harvested from E5-E9 and fixed overnight at 4° C. with 4% PFA.

### 16.7 Statistics

All results are reported as mean±standard error. The mean of each group is computed from measurements collected from at least two independent experiments. Statistical significance from in vitro experiments was determined by using a one-way analysis of variance with block (ANOVA), which was followed by Tukey's or Tukey-Kramer's multiple comparisons test (SAS 9.3). In vivo quantification used Welch's t-tests (GraphPad Software). p-values below 0.05 were considered statistically significant.

### 16.8 Tol2 Vectors Showed High Level of miRNA Expression In Vitro

Since overexpression of miR-9 by RCAS was underwhelming, alternative approaches were tested for delivery of miR-9. As an alternative strategy, pME-MCS-sd-miR9-sa was subcloned into a Tol2 vector, pT2K-CAGGS-EGFP, using Gateway (Invitrogen). Tol2 vectors can integrate into host cells in the presence of Tol2 transposase (provided by Dr. Yoshiko Takahashi; (Sato et al., 2007). By co-electroporating this construct with a vector containing a transposase (pCAGGS-T2TP), both the EGFP coding sequence and the intron containing the miRNA, which together are flanked by two Tol2 sites, will be randomly integrated into the chicken genome allowing for stable overexpression of miR-9. Previous work has shown that electroporation of a Tol2-miRNA-containing construct into the chicken otocyst produced detectable ectopic expression of the miRNA-183 family in the chicken basilar papilla (Zhang and Fekete, unpublished). This suggests that the delivery mechanism is appropriate for abundant, detectable overexpression of miRNAs in vivo.

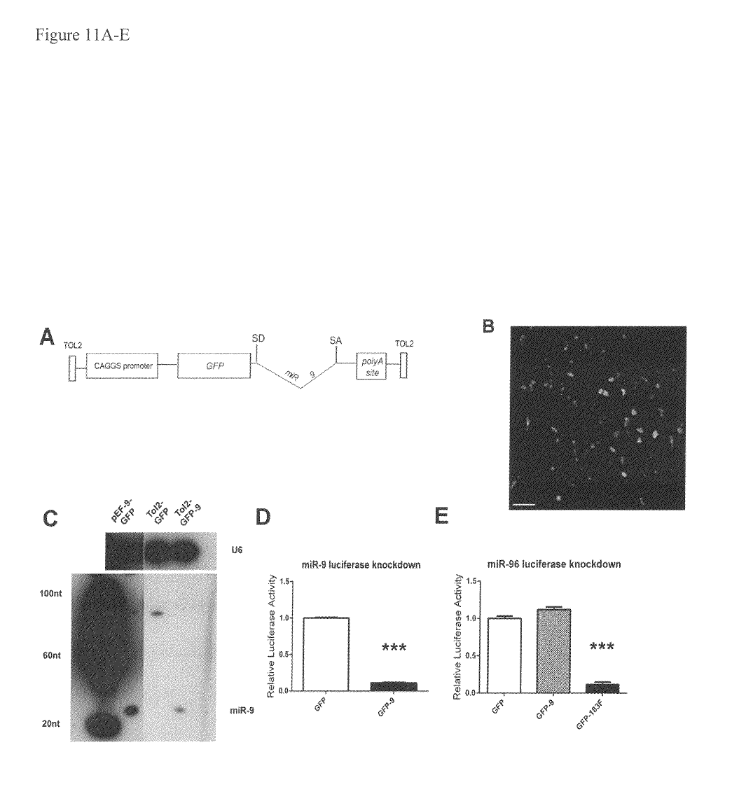

To assess the processing of miR-9 from the newly constructed pT2K-CAGGS-EGFP-9 (henceforth referred to as Tol2-GFP-9), HEK293 Ts were transfected with the vector and ˜30 hours later, the small RNA was harvested. Northern blots containing these samples were compared to controls (small RNA samples harvested from cells transfected with Tol2-GFP). Cells containing the miR-9 expression vector produced mature miR-9 (23 nucleotides) while the control sample, cells transfected with Tol2-GFP, did not (FIG. 11C).

Knowing that mature miR-9 is produced from the vector, the next step was to test its bioactivity by using the luciferase procedure discussed in Example 5 in DF1 cells. If the miR-9 being produced is functional, it should bind to its complementary sequence in the miR-9 luciferase reporter and cause a decrease in luminescence compared to the control. Indeed, a significant decrease in luminescence was seen when cells were transfected with the Tol2-GFP-miR9 vector versus Tol2-GFP (89% knockdown, p<0.0001) (FIG. 11D). Thus, it appears that the miR-9 produced is functional.

The binding specificity of miR-9 was also confirmed using luciferase assays. Luminescent ratios of cells co-transfected with a miR-96 reporter and a Tol2 vector (Tol2-GFP, Tol2-GFP-miR9, or Tol2-GFP-miR183F) were compared. If the miR-9 produced from the vector does not randomly bind to sequences present within reporter constructs, then co-transfection of the miR-96 reporter and Tol2-GFP-9 should not reduce luminescence compared to the negative control (cells co-transfected with the miR-96 reporter and Tol2-GFP). Indeed, there was no significant decrease in luminescence. In fact, there was an increase, albeit not statistically significant (12%, p=0.0715). However, cells co-transfected with Tol2-GFP-183F and the reporter showed a significant decrease in comparison to the control (89% knockdown, p<0.0001) (FIG. 11E).

### 16.9 Tol2-GFP-9 Produced Detectable Expression of miR-9 In Vivo

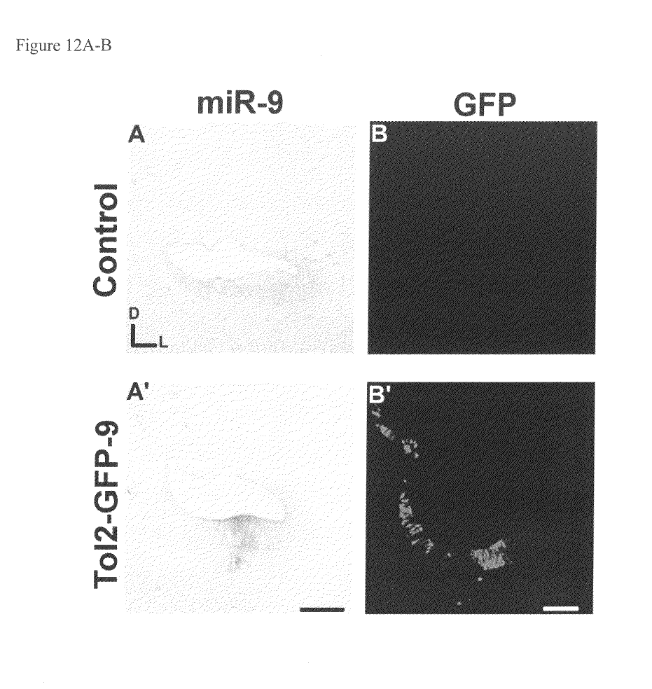

To assess whether Tol2-GFP-9 can produce GFP and miR-9 in vivo, the otocysts of E3 embryos were electroporated to drive the vector towards the posterior or anterior cristae. FIG. 12B shows the anterior cristae of an E7 embryo electroporated with Tol2-GFP-9. GFP expression is clearly detectable on the electroporated side, while the contralateral side shows lower levels. In situ hybridization performed on sister sections (FIG. 12B-B′) showed that the areas expressing GFP in the right anterior crista corresponded to overexpression of miR-9. Of the four embryos electroporated with Tol2-GFP-9 and assessed for miR-9 overexpression by in situ hybridization, all four embryos showed noticeable overexpression of miR-9 in locations that corresponded to high levels of GFP expression on adjacent sections. Therefore, the Tol2-GFP-9 vector can express miR-9 at moderate levels in vivo, and this expression localizes to the areas expressing GFP.

### CITATIONS

Bonev, B., Stanley, P., and Papalopulu, N. (2012). MicroRNA-9 Modulates Hes1 ultradian oscillations by forming a double-negative feedback loop. Cell Rep. 2, 10-18.
Chang, W., Lin, Z., Kulessa, H., Hebert, J., Hogan, B. L. M., and Wu, D. K. (2008). Bmp4 is essential for the formation of the vestibular apparatus that detects angular head movements. PLoS Genet. 4, e1000050.
Friedman, L. M., Dror, A. a, Mor, E., Tenne, T., Toren, G., Satoh, T., Biesemeier, D. J., Shomron, N., Fekete, D. M., Hornstein, E., et al. (2009). MicroRNAs are essential for development and function of inner ear hair cells in vertebrates. Proc. Natl. Acad. Sci. U.S.A. 106, 7915-7920.
Hamburger, V., and Hamilton, H. L. (1951). A series of normal stages in the development of the chick embryo. J. Embryol. Exp. Morphol. 88, 49-92.
Izumikawa, M., Minoda, R., Kawamoto, K., Abrashkin, K. a, Swiderski, D. L., Dolan, D. F., Brough, D. E., and Raphael, Y. (2005). Auditory hair cell replacement and hearing improvement by Atoh1 gene therapy in deaf mammals. Nat. Med. 11, 271-276.
Izumikawa, M., Batts, S., Miyazawa, T., Swiderski, D. L., and Raphael, Y. (2008). Response of the flat cochlear epithelium to forced expression of Atoh1. Hear. Res. 240, 52-56.
Karali M, Manfredi A, Puppo A, Marrocco E, Gargiulo A, Allocca M, Corte M D, Rossi S, Giunti M, Bacci M L, Simonelli F, Surace E M, Banfi S, Auricchio A. (2011). MicroRNA-restricted transgene expression in the retina. PLoS One. 6, e22166.
Kilpatrick, L., Li, Q., Yang, J., Goddard, J. C., Fekete, D. M., and Lang, H. (2011). Adeno-associated virus-mediated gene delivery into the scala media of the normal and deafened adult mouse ear. Gene Ther. 18, 569-578.
Kuhn, S., Johnson, S. L., Furness, D. N., Chen, J., Ingham, N., Hilton, J. M., Steffes, G., Lewis, M. a, Zampini, V., Hackney, C. M., et al. (2011). miR-96 regulates the progression of differentiation in mammalian cochlear inner and outer hair cells. Proc. Natl. Acad. Sci. U.S.A. 108, 2355-2360.
Kwan, K. M., Fujimoto, E., Grabher, C., Mangum, B. D., Hardy, M. E., Campbell, D. S., Parant, J. M., Yost, H. J., Kanki, J. P., and Chien, C.-B. (2007). The Tol2kit: a multisite gateway-based construction kit for Tol2 transposon transgenesis constructs. Dev. Dyn. 236, 3088-3099.
Lai, C. M. A. Y., Lai, Y. K. Y., and Rakoczy, P. E. (2002). Adenovirus and Adeno-Associated Virus Vectors. DNA Cell Biol. 21, 895-913.
Lewis, M., Quint, E., Glazier, A. M., Fuchs, H., De Angelis, M. H., Langford, C., van Dongen, S., Abreu-Goodger, C., Piipari, M., Redshaw, N., et al. (2009). An ENU-induced mutation of miR-96 associated with progressive hearing loss in mice. Nat. Genet. 41, 614-618.
Lin, S.-L., and Ying, S.-Y. (2004). New Drug Design for Gene Therapy—Taking Advantage of Introns. Lett. Drug Des. Discov. 1, 256-262.
Oshima, K., Shin, K., Diensthuber, M., Peng, A. W., Ricci, A. J., and Heller, S. (2010). Mechanosensitive hair cell-like cells from embryonic and induced pluripotent stem cells. Cell 141, 704-716.
Sato, Y., Kasai, T., Nakagawa, S., Tanabe, K., Watanabe, T., Kawakami, K., and Takahashi, Y. (2007). Stable integration and conditional expression of electroporated transgenes in chicken embryos. Dev. Biol. 305, 616-624.
Sheffield, A. M., Gubbels, S. P., Hildebrand, M. S., Newton, S. S., Chiorini, J. a, Di Pasquale, G., and Smith, R. J. H. (2011). Viral vector tropism for supporting cells in the developing murine cochlea. Hear. Res. 277, 28-36.
Shibata, M., Kurokawa, D., Nakao, H., Ohmura, T., and Aizawa, S. (2008). MicroRNA-9 modulates Cajal-Retzius cell differentiation by suppressing Foxg1 expression in mouse medial pallium. J. Neurosci. 28, 10415-10421.
Staecker, H., Li, D., O'Malley, B. W., and Van De Water, T. R. (2001). Gene expression in the mammalian cochlea: a study of multiple vector systems. Acta Otolaryngol. 121, 157-163.
Stoller, M. L., Chang, H. C., and Fekete, D. M. (2013). Bicistronic gene transfer tools for delivery of miRNAs and protein coding sequences. Int. J. Mol. Sci. 14, 18239-18255.
Stone, I. M., Lurie, D. I., Kelley, M. W., and Poulsen, D. J. (2005). Adeno-associated virus-mediated gene transfer to hair cells and support cells of the murine cochlea. Mol. Ther. 11, 843-848.
Torre, A. La, Georgi, S., and Reh, T. (2013). Conserved microRNA pathway regulates developmental timing of retinal neurogenesis. Proc. Natl. Acad. Sci. USA 110, E2362-70.
Weston, M. D., Pierce, M. L., Rocha-Sanchez, S., Beisel, K. W., and Soukup, G. a (2006). MicroRNA gene expression in the mouse inner ear. Brain Res. 1111, 95-104.
Zhao, C., Sun, G. G., Li, S., and Shi, Y. (2009). A feedback regulatory loop involving microRNA-9 and nuclear receptor TLX in neural stem cell fate determination. Nat. Struct. Mol. Biol. 16, 365-371.
Zine, a, Aubert, a, Qiu, J., Therianos, S., Guillemot, F., Kageyama, R., and de Ribaupierre, F. (2001). Hes1 and Hes5 activities are required for the normal development of the hair cells in the mammalian inner ear. J. Neurosci. 21, 4712-4720.

What is claimed is:

1. A microRNA (miRNA) expression vector comprising a promoter sequence and an artificial intron, wherein the promoter sequence is positioned upstream (5′) of the artificial intron, and wherein the artificial intron comprises a nucleic acid sequence encoding one or more miRNA genes.

2. The vector of claim 1, wherein said promoter is a RNA polymerase II-based promoter.

3. The vector of claim 1, wherein said promoter is elongation factor-1 alpha (EF1α) promoter.

4. The vector of claim 1, wherein the one or more miRNA genes is selected from the group consisting of MIR9, MIR96, MIR182, and MIR183.

5. The vector of claim 1, wherein the one or more miRNA genes is each of MIR96, MIR182, and MIR183.

6. The vector of claim 1, wherein the miRNA expression vector is a viral vector or a plasmid.

7. The vector of claim 6, wherein the viral vector is selected from the group consisting of an adenovirus, a retrovirus, an adeno-associated virus, and a herpes simplex virus.

8. The vector of claim 1, wherein the vector further comprises an exogenous gene, positioned either between the promoter and the artificial intron or downstream of the artificial intron.

9. The vector of claim 8, wherein the exogenous gene encodes a reporter, a selectable marker or other functional protein.

10. The vector of claim 9, wherein the reporter, selectable marker or other functional protein is fused to a hemagglutinin epitope tag.

11. A method of altering expression of one or more genes in a cell, comprising introducing into a cell an miRNA expression vector of claim 1, wherein the expression of said one or more genes is altered.

12. The method of claim 11, wherein said promoter is a RNA polymerase II-based promoter.

13. The method of claim 11, wherein said promoter is elongation factor-1 alpha (EF1α) promoter.

14. The method of claim 11, wherein the one or more miRNA genes is selected from the group consisting of MIR9, MIR96, MIR182, and MIR183.

15. The method of claim 11, wherein the one or more miRNA genes is each of MIR96, MIR182, and MIR183.

16. The method of claim 11, wherein the miRNA expression vector is a viral vector selected from the group consisting of an adenovirus, a retrovirus, an adeno-associated virus, and a herpes simplex virus.

17. The method of claim 11, wherein the vector further comprises an exogenous gene, positioned either between the promoter and the artificial intron or downstream of the artificial intron, wherein the exogenous gene encodes a reporter, a selectable marker or other functional protein.

18. A method of treating hearing impairment in a subject, comprising administering to a subject in need thereof an effective amount of an miRNA expression vector comprising a promoter sequence, an artificial intron and a nucleic acid sequence encoding a transcription factor, wherein the promoter sequence is positioned upstream (5′) of the artificial intron and the nucleic acid sequence encoding the transcription factor, wherein the artificial intron comprises a nucleic acid sequence encoding one or more miRNA genes, wherein the transcription factor promotes expression of a silenced gene, and wherein hearing impairment is treated in the subject.

19. The method of claim 18, wherein the promoter is elongation factor-1 alpha (EF1α) promoter.

20. The method of claim 18, wherein the one or more miRNA genes is each of MIR96, MIR182, and MIR183.
